(19) United States
(12) Patent Application Publication
(10) Pub. No.: US 2023/0220354 A1
(43) Pub. Date: Jul. 13, 2023

(54) COMPOSITIONS AND METHODS FOR CONTROLLING CELLULAR IDENTITY

(71) Applicants: King Abdullah University of Science and Technology, Thuwal (SA); Salk Institute for Biological Studies, La Jolla, CA (US)

(72) Inventors: Pierre Julius Magistretti, Thuwal (SA); Juan Carlos Izpisua Belmonte, La Jolla, CA (US); Reyna Hernandez Benitez, La Jolla, CA (US)

(21) Appl. No.: 17/995,152

(22) PCT Filed: Mar. 30, 2021
(86) PCT No.: PCT/US2021/024938

§ 371 Date: Sep. 30, 2022

Related U.S. Application Data
(60) Provisional application No. 63/002,063, filed on Mar. 30, 2020.

(51) Int. Cl. C12N 5/074

(52) U.S. Cl. CPC C12N 5/0696; C12N 2500/32; C12N 2500/99

(57) ABSTRACT

Compositions and methods modulating the steady state of cells are provided. The compositions include metabolites (C1 metabolites and C1 metabolite cocktails (C1-MIM) for use in inducing cells into a different state from their steady state, for example, into a less differentiated state, when compared to their original state before treatment. The C1 metabolites include methionine, SAM (S-adenosyl methionine), threonine, glycine, putrescine, and cysteine. The metabolites are used to supplement cell culture media, and accordingly, cells culture media supplemented with the disclosed metabolites (MIM supplemented media) are also provided.

The method includes: contacting a cell with the C1 metabolites for a sufficient period of time to result in reprograming the cell into a different state from their steady, for example, into a less differentiated state having progenitor-like characteristics (MIM-Cells). Isolated MIM-cells and their progeny, can be used in a number of applications, including cell therapy and tissue engineering.

### CROSS-REFERENCE TO RELATED APPLICATION

The present application claims priority to U.S. Application No. 63/002,063, filed Mar. 30, 2020, the disclosure of which are incorporated herein by reference in their entirety.

### FIELD OF THE INVENTION

The present invention generally relates to compositions and methods for reprograming eukaryotic cells from their steady state, into a different cellular state.

### BACKGROUND OF THE INVENTION

Metabolomic analyses reveal the specific array of metabolites present in a cell type at any given time. So far there is little evidence of whether metabolic switches and specific metabolites are drivers of changes in cellular identity. Previous studies which focused on investigating the metabolomic dynamics of cellular differentiation by assessing cell state progression using long term time points, on the scale of days (Tormos, et al. Cell Metab. 14, 537-544 (2011); Panopoulos, et al. Cell Res. 22, 168-177 (2012); Park, et al. Neurosci. Lett. 506, 50-54 (2012); Bracha, et al. Nat. Chem. Biol. 6, 202-204 (2010)), miss critical metabolic changes associated with (or potentially driving) the very earliest transitional steps from one cell phenotype to another, and which could be modulated to control cell fate. New methods and compositions are needed to reprogram and control cell fate.

It is an object of the present invention to provide compositions for reprograming cells from their steady state, into a different cellular state.

It is a further object of the present invention to provide methods for reprograming cells from their steady state into a different cellular state.

It is also an object of the present invention to provide cells reprogrammed from their original steady state, into a different cellular state.

### SUMMARY OF THE INVENTION

Compositions and methods for reprogramming cells from their steady state into a different cellular state, are provided. In some embodiments the compositions and methods are for de-differentiating differentiated or partially differentiated cells.

In other embodiments, the compositions and methods are for differentiating non-terminally differentiated cells. The compositions include metabolites (C1 metabolites and C1 metabolite cocktails (C1-MIM) for use in inducing cells into a less differentiated state, when compared to their original state before treatment. The C1 metabolites include methionine, SAM (S-adenosyl methionine), threonine, glycine, putrescine, and cysteine. The metabolites are used to supplement cell culture media, and accordingly, cells culture media supplemented with the disclosed metabolites (MIM supplemented media) are also provided.

Methods for reprogramming cells from their steady state into a different cellular state, for example, de-differentiating differentiated or partially differentiated cells are provided. The method includes culturing differentiated partially differentiated or non-differentiated steady state cell in an MIM supplemented medium for a period of time effective to change its steady state. In some preferred embodiments, the MIM supplemented medium includes six C1 metabolites. In some preferred embodiments the MIM supplemented includes methionine threonine and glycine, putrescine, and most preferably, includes no serum or reduced serum (less than 3%) and/or a survival factor such as FGFs.

Also disclosed are cells chemically reprogramed with C1 metabolites (MIM-Cells). In one preferred embodiment, the cells are obtained following culture in the MIM supplemented cell culture media, supplemented with effective amount of the metabolite to reprogram the cells by reversing their state of differentiation into a less differentiated state, and a progenitor-like state, characterized in a reduction of at least one mature cell marker and an upregulation in the expression of at least one genes characteristic of a progenitor state.

The disclosed MIM cells can be used in cell therapy and tissue engineering applications.

### BRIEF DESCRIPTION OF THE DRAWINGS

FIGS. 1A-1D show identification of the early transitions on the gene expression between two cellular phenotypes. FIG. 1A on the left panel, the rationale for the selection of time window of interest, according to the gene expression profile of markers that identify the transition between two cell phenotypes during normal differentiation. On the right panel, experimental overview for exploring the metabolome, focusing in the intermediate transcriptional populations differentiated from Myoblasts (MB s), Neural Stem Cells (NSCs), and Mesenchymal Stem Cells (MSCs), i.e., the populations crossing an intermediate stage of commitment between the original and subsequent cell phenotype. FIGS. 1B-1D show gene expression profiles during differentiation of MSCs towards chondrocytes (FIG. 1B), NSCs towards astrocytes (FIG. 1C), and MBs towards myofibers (FIG. 1D), represented as percentage over time zero (time 0h=MSC-, NSC-, or MB-state). Total RNAs were extracted and the mRNA levels were detected by rtPCR. The gene expression was normalized with the geometric mean of three housekeeping genes (Actb, Gapdh and Nat1) and then normalized versus control condition (time=0h). Represented the means ±SEM, n≥3. FIG. 1E is a schematic representation of the recognized patterns regarding the abundance of individual metabolites over time (top panel). Overlapped in the bell-shaped pattern that temporally fits with the intermediate transitional populations differentiated from their respective precursor (initial cell identity). FIG. 1F. is a graph showing the rationale of the relevance of the pattern in addition to the level of metabolites. Hypothetical case of three metabolites present in the intermediate transcriptional transition. After inducing the differentiation a transitional phase of transcriptional programs occurs (central area), in that phase the three hypothetical metabolites are found. Observe that the level of metabolite-2 can be consequence only of the deactivation of the initial transcriptional program (from the steady cell type 1), despite metabolite-2 is higher than metabolite-1. Conversely, metabolite-1 and metabolite-3, only raise their levels during the intermediate transition, therefore those should have more relevance for that transcriptional transition. FIG. 1G is a bar graph showing cells types before MIM treatment. FIG. 1H shows relative gene expression of Gfap, cMyc, and Nestin against the exposure of a range of concentrations of each of the components for C1-MIM. FIG. 1I shows gene expression in response to C1-MIM. FIG. 1J shows Gfap gene expression as a result of combinations with/without SAM.

FIG. 2A.Representation of the identification of an increase in the relative levels of metabolites in three different cell populations derived from

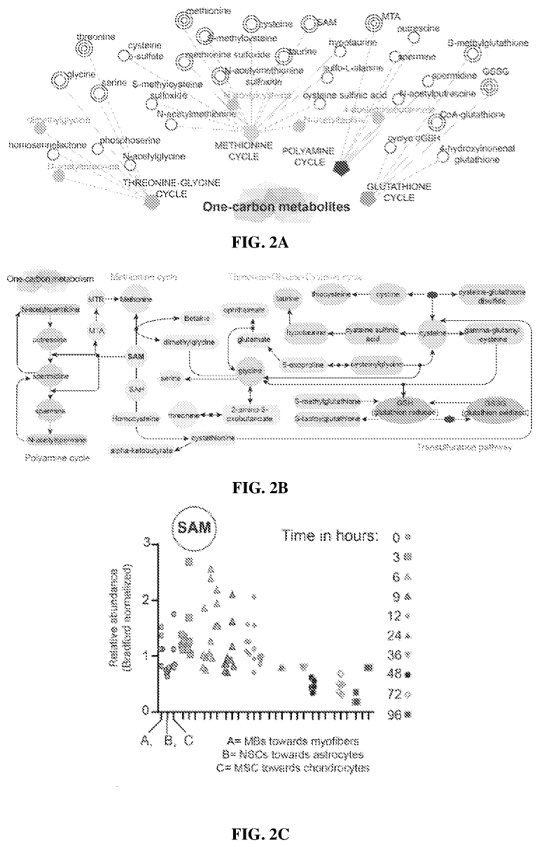

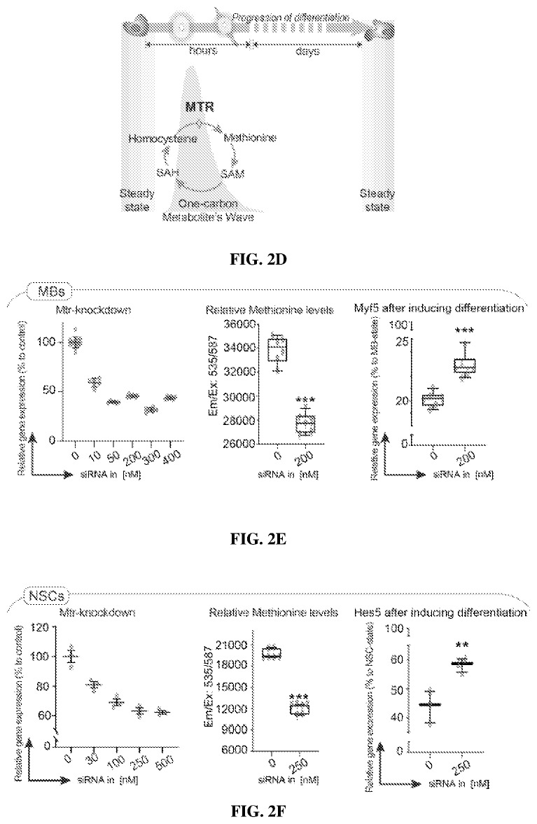

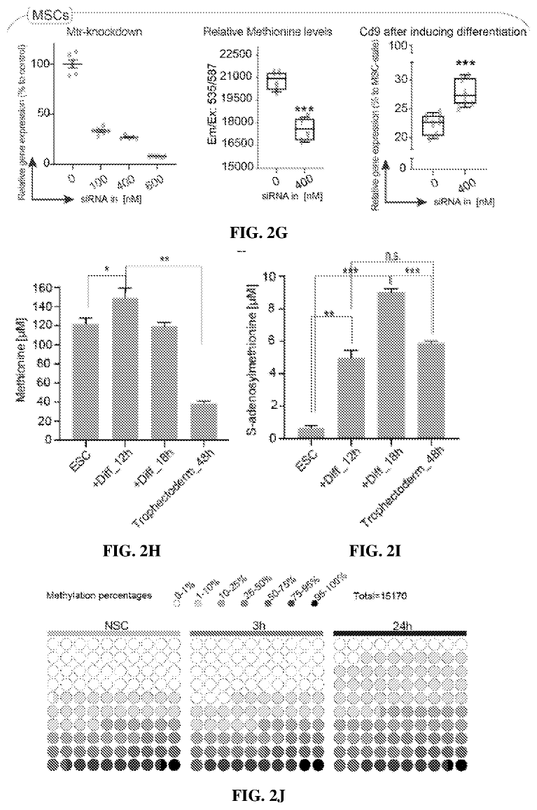

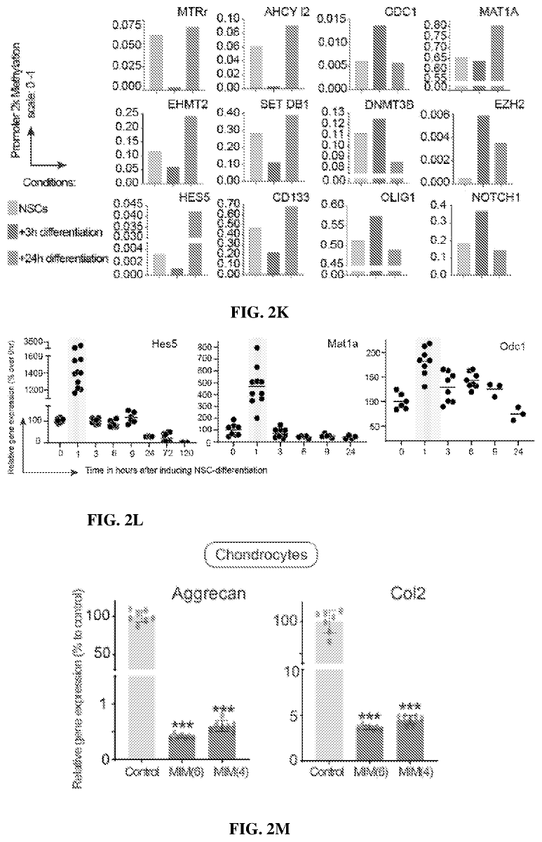

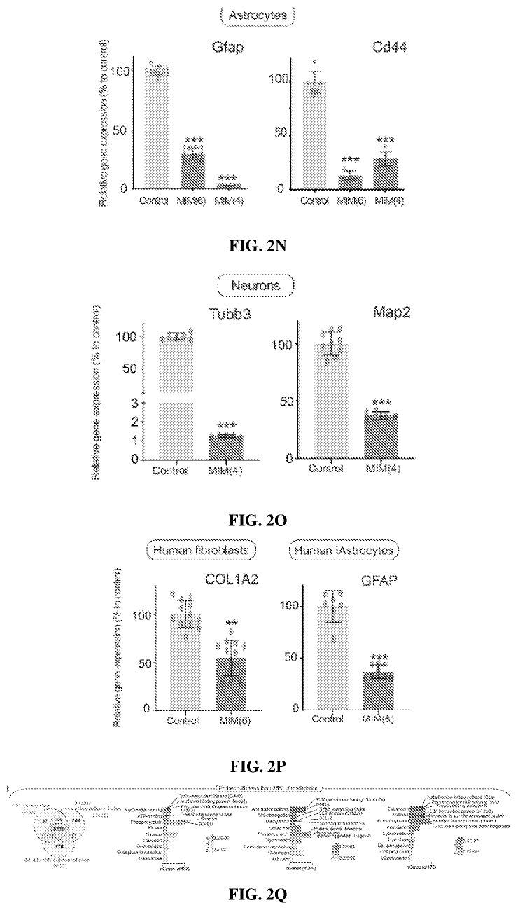

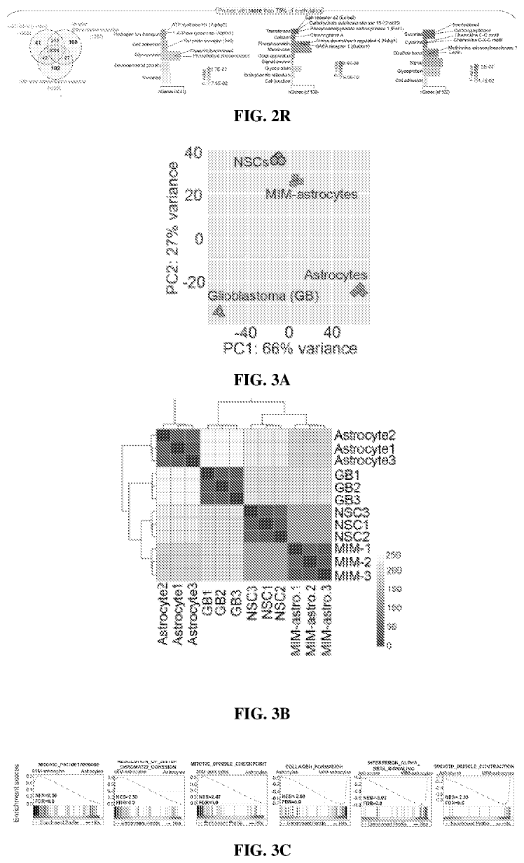

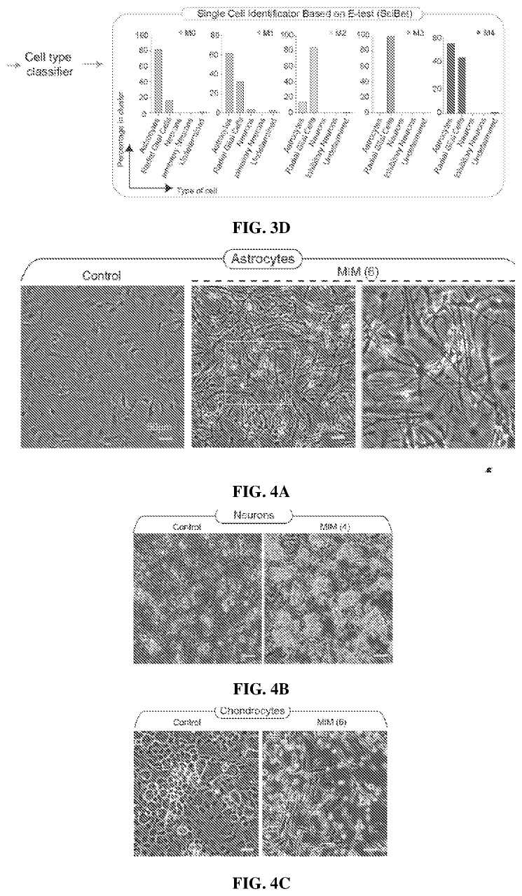

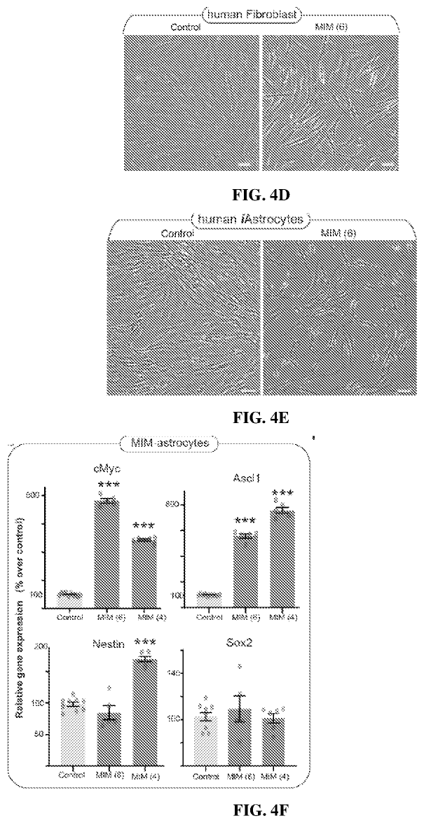

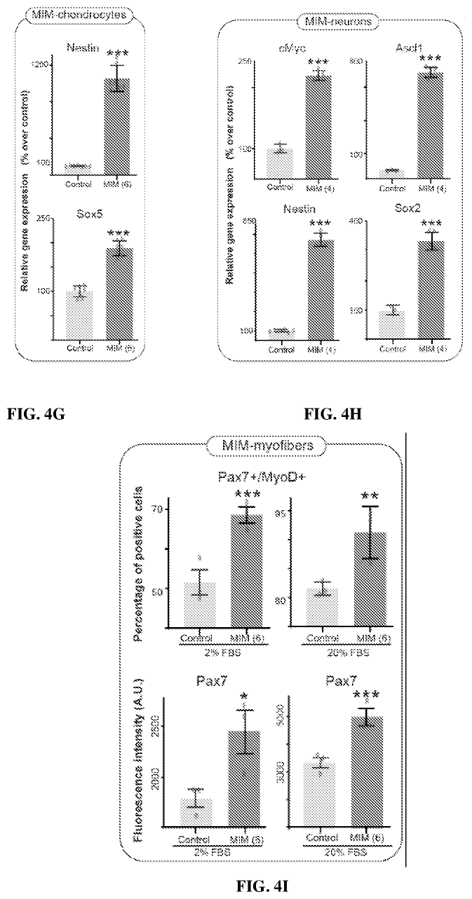

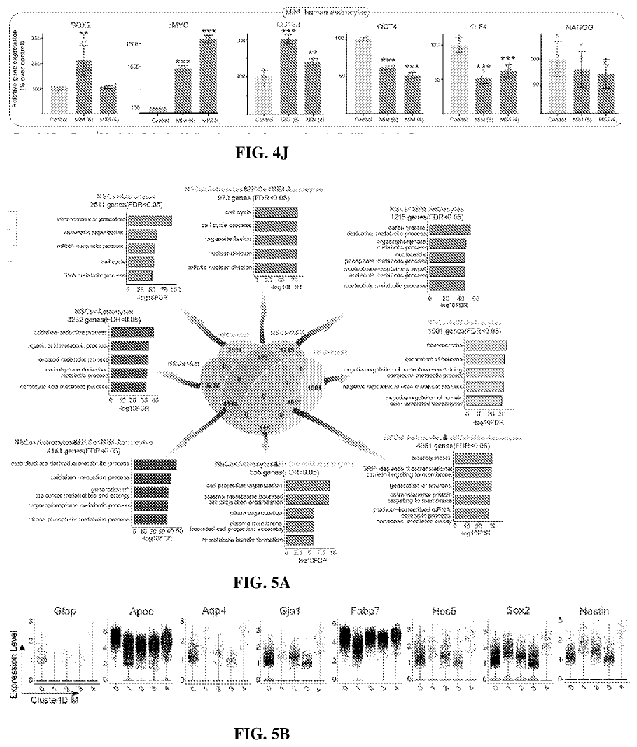

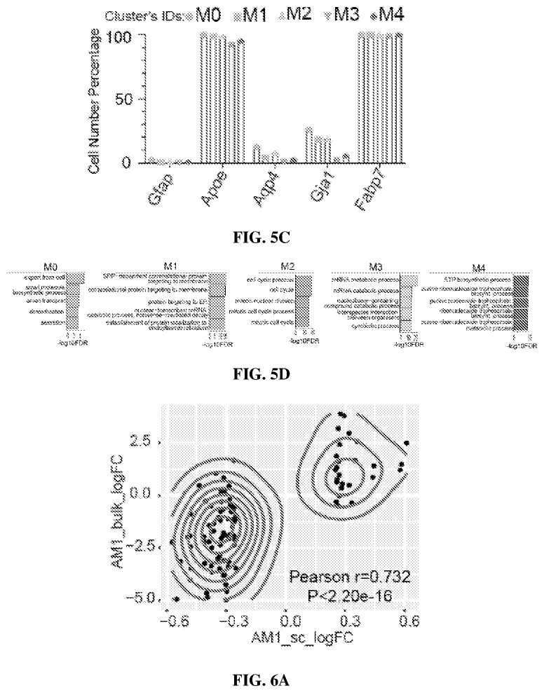

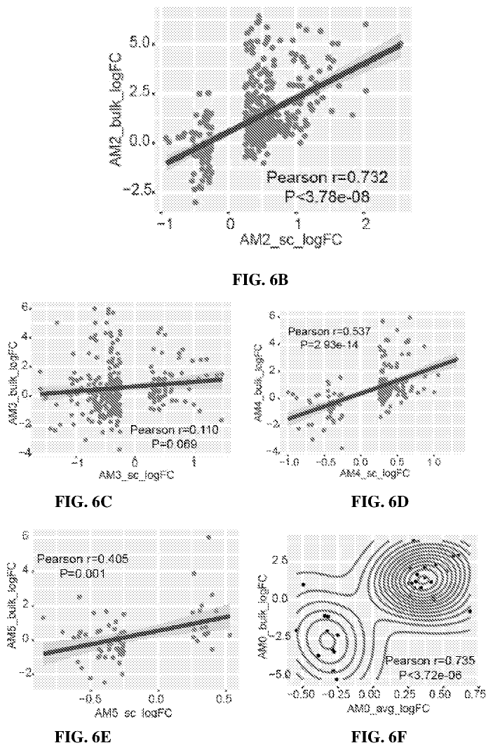

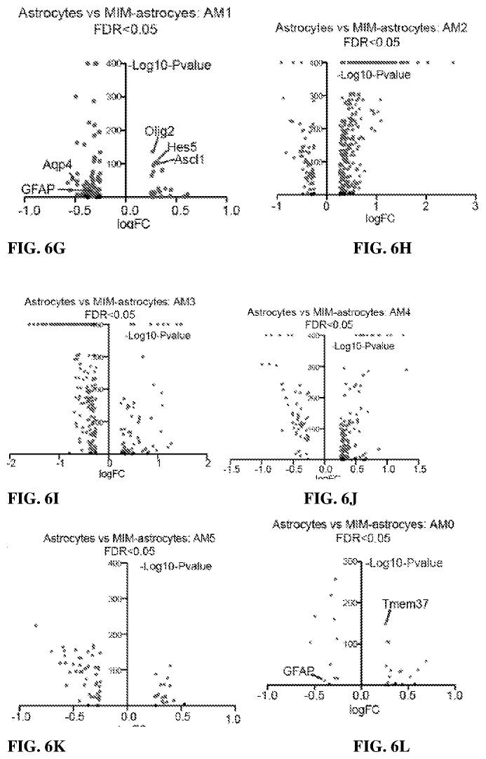

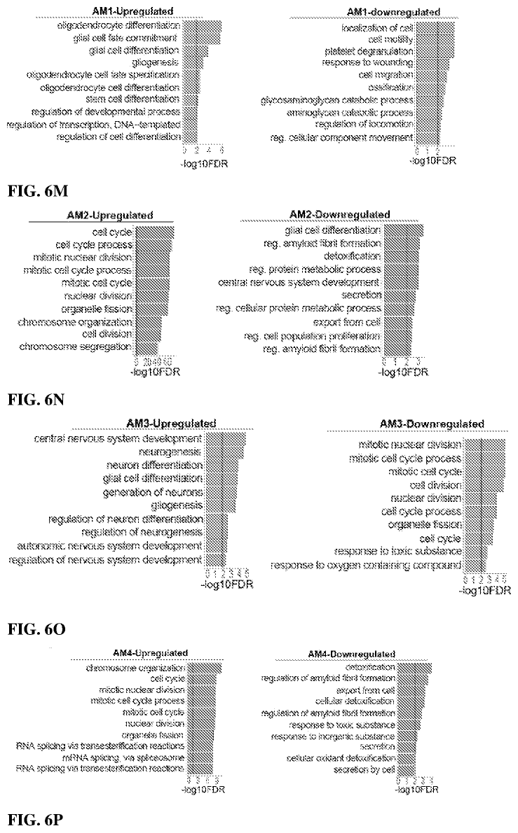

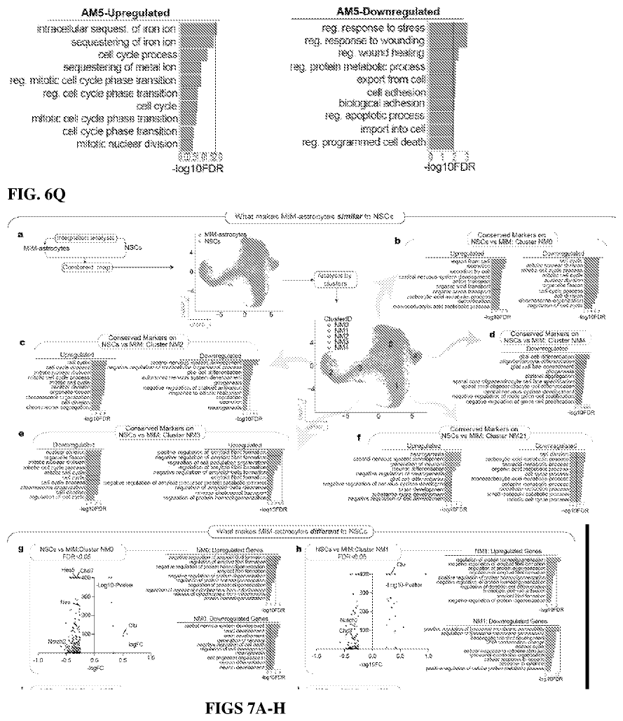

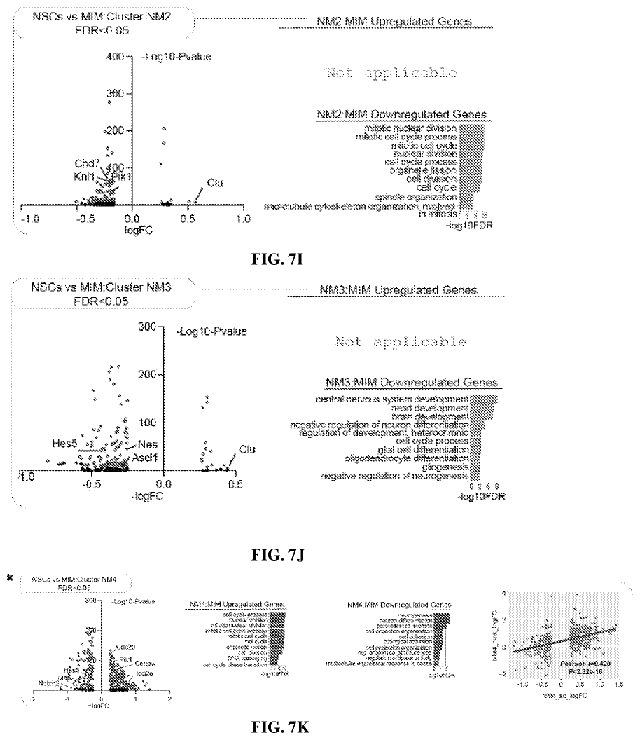

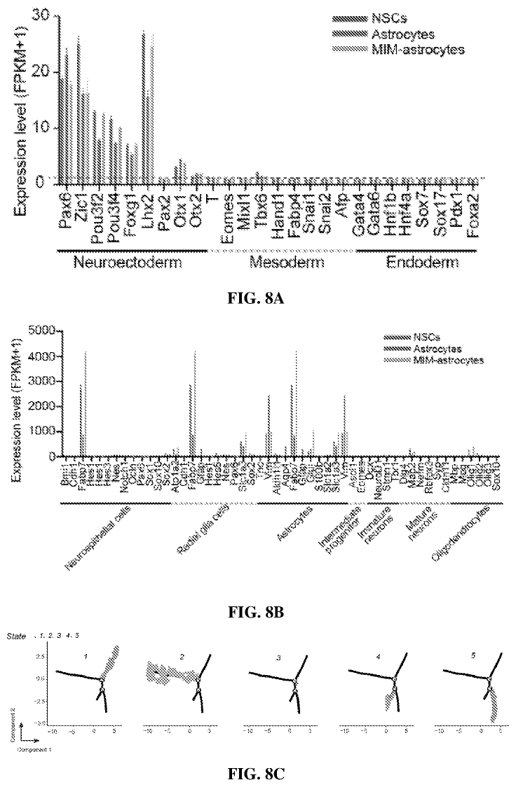

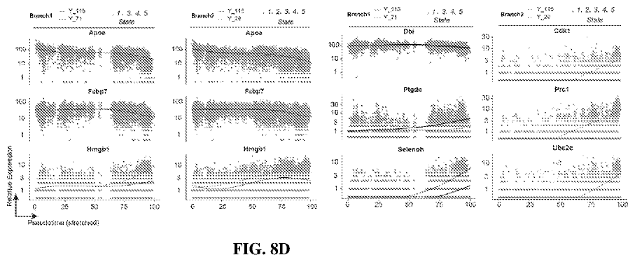

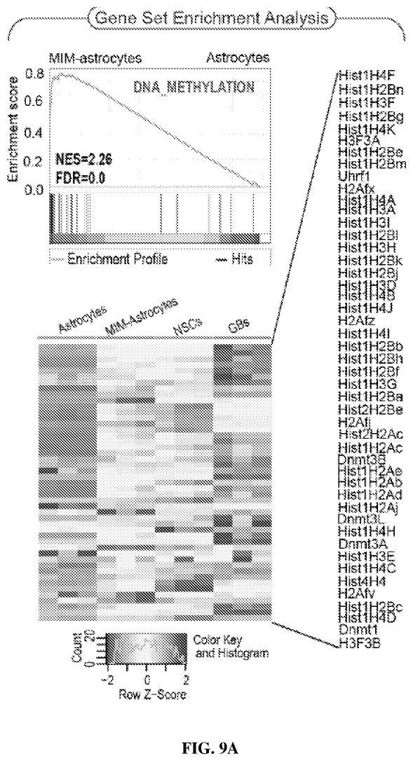

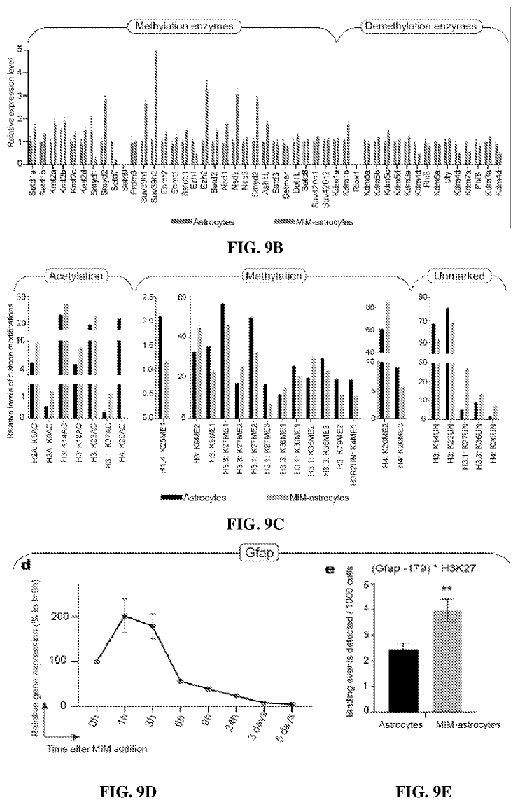

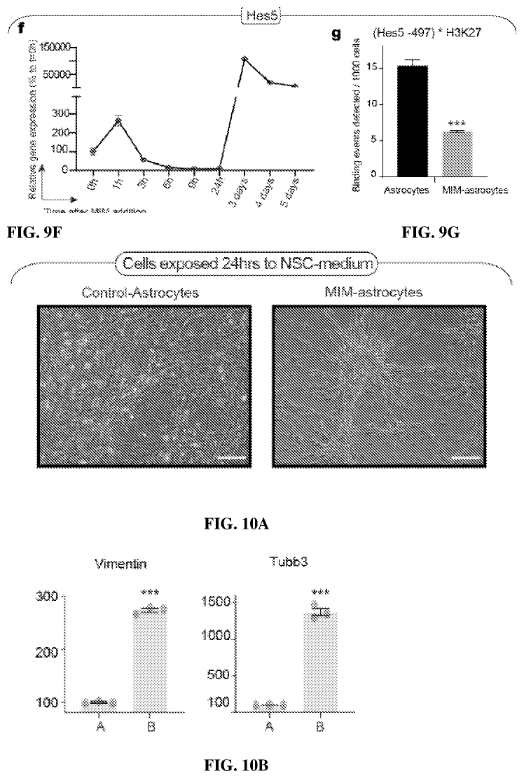

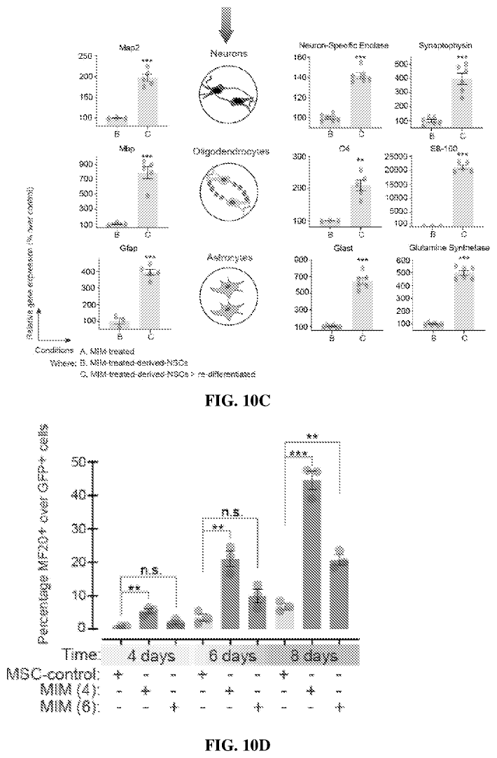

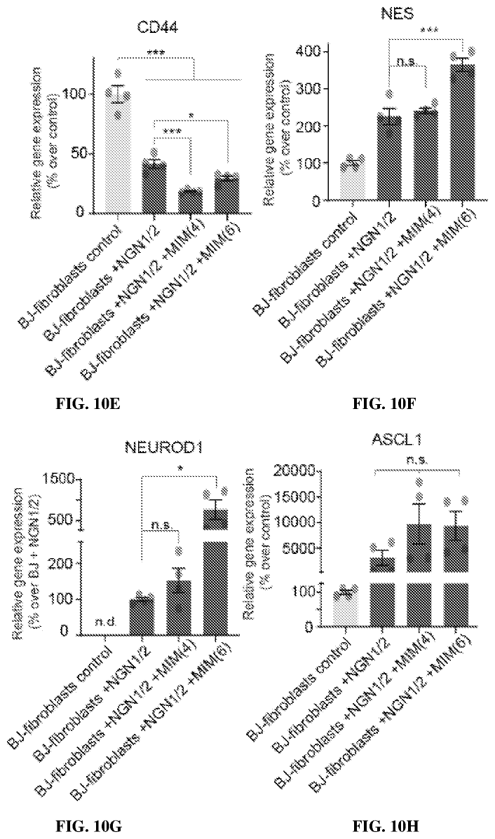

MBs, NSCs, and MSCs. Metabolites grouped in 4 categories. Open circles represent metabolites that increase significantly at 6 hours after inducing their differentiation (P<0.05). One, two, or three circles denote significant increases in one, two, or the three cell types. respectively. Gray circles represent metabolites that only increase in one cell type, while in the other two showed a negative trend. representing the main cycles belonging to the One-carbon metabolism. FIG. 2B One-carbon metabolic network. FIG. 2C. Relative abundance of S-adenosylmethionine (SAM) normalized by scaled intensity (y-axis) during differentiation of three cell types (MBs, NSCs, and MSCs). The scaled intensity observed was Bradford-normalized and represents the relative levels of SAM inside the cell at the indicated times. Data shown as distribution with all values. Experiments shown are from n˜5 biological replicates per cell lineage. FIG. 2D. Schematic representation of the timing associated with a wave of One-car bon-metabolites during the change in identity between two steady-states. Represented the methionine cycle in blue arrows, and one key enzyme (yellow square) of this cycle. Note scales: hours vs. days.. FIG. 2E-2G. Mtr-knockdown and disturbance of One-carbon wave in MBs in if FIG. 2E. NSCs in FIG. 2F and MSCs FIG. 2G. On the left panels, siRNA-Mtr dose-response showing the degree of inhibition of Mtr gene expression in each cell type. Transfection of siRNA performed at the multipotent stage; knockdown measured one day after. On the middle pane 1s, the quantification of relative levels of methionine (immediate metabolite affected by MtT-knockdown). Quantification done by a fluorescence method; dots represent absolute values measured at emission/excitation (EnVEx) 535/587. On the right panels, relative gene expression of a marker of the multipotent stage for each cell type, measured in knockdown cells after inducing differentiation (with a selected siRNA-Mtr concentration). Gene expression obtained by rtPCR and normalized with the geometric mean of two housekeeping genes (Actb) and Gapdh); then normalized to control condition as 100% to observe the percentage of inhibition or potentiation. Means±SEM, statistically significant from controls at **P<0.01, ***P<0.001 and n>/=3, where dots represent each value, FIG. 2H. Quantification of methionine levels on early states after inducing differentiation of ESCs to trophectoderm (in the cell line Zhbtc4);

n˜4 technical replicates, significantly different at *P<0.05, **P<0.01 (paired t-test). FIG. 2I. Quantification of S-adenosylmethionine (SAM) levels on early states after inducing differentiation of ESCs to trophectoderm (in the cell line Zhbtc4); n˜4 technical replicates, significantly different with **P<O.OI, ***P<O.OOI (paired t-test). FIG. 2J. Proportions of methylation percentages ofPromoter2K regions (0-2 ,000 bp upstream of transcription start site) separated by seven ranges considering a total of 15170 probes. FIG. 2K. Representative genes with differential methylation percentages ofPrornoter2K regions. FIG. 2L. Relative gene expression of the indicated genes during early times after inducing differentiation of NSCs. Total RNAs were extracted and the mRNA levels were detected by rtPCR. Here an oscillatory peak in gene expression is indirectly observed by rt-PCR, when occurs a higher variation in the relative levels of gene expression reached by different pools of cells crossing by the same time. Note such variation is observed specifically at one timing; besides other genes evaluated using the same samples do not exhibit such variation (see discussion SI-1e). Gene expression normalized with the geometric mean of two housekeeping genes (Acid) and Gapdh) and then normalized vs. control condition (time Oh˜NSCs). Each dot represents one sample. FIG. 2M-P. Gene expression by rtPCR of the main differentiation markers of different cell types—indicated—in control differentiation media (light gray bars) and treated cells with different combinations of MIM-components (dark bars). Where: MiM(6) contains Methionine, Glycine, Putrescine, Cysteine, S-adenosylmethionine; while the MIM(4), the former composition minus Cysteine and minus S-adenosylmethionine. All metabolites are meant to feed the C I-network, details in discuss ion f. The expression was normalized with the geometric mean of three housekeeping genes (Actb, Gapdh and Nati , for mouse cells; GAPDH, RI,PI9 and GUS for human induced astrocytes; and GAPDH for human fibroblasts) and then normalized vs. control condition. Bars are the mean±SEM, statistically significant from controls at **P<0.01 or ***P<0.001; and n>/=5, where dots represent each value. FIG. 2Q. Comparison of Promoter2K regions having methylation percentages below 25%. First panel shows the overlap of RefSeg accession numbers associated with each condition. Second to fourth panels, functional enrichment analyses of genes that exclusively show less methylation for each condition (NSC, 3h, or 24h). FIG. 2R. Comparison ofPromoter2K regions having methylation percentages above 75%. First panel shows the overlap of RefSeq accession numbers associated with each condition. Second to fourth panels, functional enrichment analyses of genes that exclusively show more methylation for each condition (NSC, 3h, or 24h).

FIG. 3A shows PCA map of astrocytes, MIM-astrocytes, NSCs and Glioblastoma (GB) based on the gene normalized expression level. The transcriptomic profile was obtained by bulk RNAseq; each sample is represented by single dots (n=3). FIG. 3B Heat map of Euclidean distance of all the samples based on the calculation from the regularized log transformation. FIG. 3C. Gene set enrichment analysis (GSEA) of the DEGs between astrocytes and MIM-astrocytes. Panel shows three top enrichment distribution associated with upregulated and downregulated genes in MIM astrocytes. FDR and the normalized enrichment score (NES) shown for each plot. FIG. 3D. Machine learning prediction of the cell types identified after treatment with MIM in astrocytes. Predicted from development neuron datasets, according to the atlases of the Since Cell Identifier Based on E-test (SciBet).

FIG. 4A-E. Representative images of cultures control and five days after the treatment with MIM. Scale bar, 50llm. The yellow square in (FIG. 4A) represents a digital amplification of the selected area, Arrowheads in the amplified area show cells with acquired morphology after MIM-treatment, in the middle of cells that kept control morphology. FIG. 4F-4H, and 4J. Relative gene expression obtained by rtPCR in control and MIM-treated cells with different combinations of MIM-components. Where: MIM (6) contains Methionine, Glycine, Putrescine, Cysteine, S-adenosylmethionine; while the MIM (4), the former composition minus Cysteine and minus S-adenosylmethionine, The expression was normalized with the geometric mean of at least two housekeeping genes (from Actb, Gapdh, and Nati) and then normalized vs. control condition. Red dots represent each value (n 5), and bars the mean±SEM, where differences compared with control are significant at **P<0.001 or ***P<0.0001. FIG. 41. Relative expression ofPax7+cells evaluated by immunocytochemistry. Top panel percentage ofPax7+cells vs. total MyoD+cells. Bottom panel fluorescence intensity of the Pax7 marker. In this condition, MIM₆ was used in a media reduced in serum. Red dots represent each value. Bars are mean±SEM, where differences compared with control are significant at * I′ <0.05, **P <0.001, ***P <0.0001. Panels FIG. 4A-4C and FIG. 4F-4I are experiments in the indicated mouse cells. Panels FIG. 4D, 4E, and 4J in human cells, where iAstrocytes stands for induced-Astrocytes

FIG. 5A. Gene Ontology (GO) analysis of the overlap on differential expressed genes from NSCs vs. Astrocytes and Astrocytes vs. MIM-treated astrocytes. FIG. 5B shows gene expression levels in MIM-astrocytes. FIG. 5C. Cell number percentages of astrocytes-markers in each cluster of MIM-astrocytes. FIG. 5D. GO analysis of each cluster in MIM-astrocytes

FIG. 6A-F. Correlation of gene expression as fold-change from each cluster of single-cell RNA data and bulk RNA data, Pearson correlation values indicated in the plots. FIG. 6G-L. Volcano plots of differential expressed genes in indicated clusters (AMO-AMS) based on alignment between astrocytes and MIM-astrocytes. Red points indicating FOR<O.OS taken as significant. FIG. 6M-Q, Gene Ontology biological terms associated with each cluster of MIM-astrocytes compared to astrocytes. P-value calculated with hypergeometric tests and FDR calculated using the

Bonferroni correction procedure. Note: AM1-cluster DEG analysis was performed comparing astrocytes and MIM-astrocytes, while each other cluster from AM2-AMS (as they do not have matching clusters in astrocytes) were compared vs. the whole astrocyte population (pool composed by AMO+AMI from astrocytes).

FIG. 7A. Rationale for the figure. Computational approaches allow the integration of MIM-astrocytes and NSCs scRNAseg data. Follow the arrows to observe the combined map, which in turn is subjected to a clustering analysis to define subpopulations NMO-NW. From each cluster. we analyzed the gene expression conserved, following for Gene Ontology (GO) analysis. FIG. 7B-F. GO analyses of the conserved expression of genes between NSCs and MIM-astrocytes, including unregulated and downregulated genes for each cluster. Note: in (FIG. 7D) only downregulated enrichment due to the few number of genes upregulated this specific similarity comparison, FIG. 7G-K. On the left side, volcano plots of differential expressed genes in clusters NMO-NM4 based on the alignment between NSCs and MIM-astrocytes showed in FIG. 7A) red points indicating FOR<O.05 taken as significant. On the right side, GO analyses of differential expressed genes, including upregulated and downregulated genes from each cluster. In FIGS. 7I and 7J the enrichment is only for downregulated genes, due to a reduced number of up regulated ones, On the right side of FIG. 7K, the Pearson correlation of the fold-change gene expression from scRNA data and bulk-RNA data. N=NSCs; M=MIM-astrocytes, NM integration NSCs and MIM-astrocytes. P-values calculated with hypergeometric tests; FOR calculated using the Bonferroni correction procedure.

FIG. 8A. Identification of marker genes for neuroectoderm, mesod.erm, and endoderm lineages based on the FPKM values of bulk-RNA seq, according to the parameters considered by Nakajima-Koyama et al., (2015). FIG. 8B. Assignment of cell types present in MIM-astrocytes according to the Panglao DB interface. FIG. 8C. Characterization of transient cell states by scRNAseq trajectory analysis. Facet of the trajectory plot recognizing five states and two branches using Monocle's approach. FIG. 8D. Recognition of representative genes that change in MIM-astrocytes as function of pseudo time.

FIG. 9A. shows on the top panel, Gene Set Enrichment. Analysis (GSEA) shows the distribution set for DNA methylation in MIM-astrocytes (top panel). On the low panel, the correspondent heatmap for DNA methylation related genes based on the normalized expression values compared to astrocytes, NSCs, and glioblastoma cells (GBs). FIG. 9B shows relative expression levels of genes encoding for proteins associated with methylation and demethylation processes. Data in (a, b) derives from bulk-transcriptomics (n˜3). FIG. 9C shows histone modifications. Changes in the relative abundance of each form indicated of amino acid residue in bulk histones isolated from astrocytes and MIM-astrocytes. Bars represent the relative abundances of each form of indicated peptides, means and standard deviation of corresponding measurements from three mass spec ‘runs. Showing all the modifications significantly different between astrocytes and MIM-astrocytes. FIGS. 9D and 9F. Relative gene expression obtained by rt-PCR in astrocytes (control at time Oh) and at consecutive times after adding Ci-MIM to the cells. Gfap in (FIG. 9D) and Hes5 in (FIG. 9F). The expression was normalized with the geometric mean of two housekeeping genes (Actb and Gapdh), then normalized vs. control condition. Pink dots represent the average value±SEM, (n :>/=5). FIG. 9E and 9G. ChIP-qPCR from astrocytes and MIM-astrocytes (evaluated at day 5) for the indicated promoter ’ sites of Gfap in (FIG. 9E) and Hes5 in (FIG. 9F). Data derives from qPCR reactions set in triplicates for each ChIP sample for the methylation on H3K27. Normalized data (according input-DNA) is expressed as binding events detected per 1000 Cells (see Methods for details). Significantly different from astrocyte control at **P <0.01, *** 0.001.

FIGS. 10A-10C are functional assays of showing the capacity acquired by old cerebellar astrocytes (18.S′ months old mice) after the CI-MIM treatment. FIG. 10A shows the capacity for neurosphere-formation. Bright-field pictures of control astrocytes and MIM-astrocytes, both exposed 24h to NSCs-standard proliferation medium (scale bar˜150llm). FIG. 10B shows relative gene expression by rtl'CR in A˜MIM-astrocytes compared to B-M1M-astroCy1es-derived-NSCs. FIG. 10C shows relative gene expression of main markers for astrocytes, oligodendrocytes, and neurons, acquired after re-differentiate the MIM-astrocytes-derived-NSCs (=C). The expression was normalized with the geometric mean of two housekeeping genes (Actb and Gapdh) and then normalized vs. respective control conditions. Dots represent each value, bars are means±SEM. FIG. 10D shows percentages of MF20 positive cells over GFP expressed cells obtained by direct counting of fluorescents; dots represent each value±SEM. FIG. 10E shows relative expression of genes for fibroblast-identity in (j) and FIGS. 10 F-H show relative expression of genes fibroblast-identity in. The expression was normalized with the mean of the housekeeping gene CTCF, then normalized vs. control condition BJ-fibroblasts; except in (FIG. 10G) where NEURODI was not detected in fibroblasts and the conditions with MIM were normalized vs. BJ-fibroblast+NGN1I2. Dots represent each value±SEM. Significantly differences at *P<0.05,**P <0.005, ***P<0.0001.

### DETAILED DESCRIPTION OF THE INVENTION

Previous studies have compared the metabolomes of stable, committed cellular states, or tracked metabolomic changes during differentiation using large time windows that did not capture initial transcriptional changes (Knobloch, et al. Cell Rep. 20, 2144-2155 (2017); Yanes, et al. Nat. Chem. Biol. 6, 411-417 (2010); Peng, et al. Science 354, 481-484 (2016); Castiglione, et al. Sci. Rep. 7, 15808 (2017)). These studies have primarily focused on steady-state conditions, for example, comparing stem/progenitor cells to fully differentiated cells (Yanes, et al. Nat. Chem. Biol. 6, 411-417 (2010); Wang, et al. EMBO J. 36, 1330-1347 (2017); Panopoulos, et al. Cell Res. 22, 168-177 (2012); Coller, FEBS Lett. 593, 2817-2839 (2019))). Previous studies have investigated the metabolomic dynamics of cellular differentiation by assessing cell state progression using long term time points, on the scale of days (Tormos, et al. Cell Metab. 14, 537-544 (2011); Moussaieff, et al. Cell Metab. 21, 392-402 (2015).; Guijas, et al. Nat. Biotechnol. 36, 316-320 (2018)).

The compositions and method disclosed herein are based on studies conducted to examine on the metabolomic changes occurring during the early phases of in vitro cell differentiation in three different multipotent stem cell types (myoblasts, MBs; neural stem cells, NSCs; and mesenchymal stem cells, MSCs), which uncovered the existence of specific waves of metabolites coupled to the transition of transcriptional programs necessary to drive forward cellular differentiation. These conserved metabolic waves can be engineered to reverse a cell's steady state, for example, cell differentiation, and thus be utilized to induce cellular plasticity.

### I. Defintions

“Culture” means a population of cells grown in a cell culture medium and optionally passaged. A cell culture may be a primary culture (e.g., a culture that has not been passaged) or may be a secondary or subsequent culture (e.g., a population of cells which have been subcultured or passaged one or more times).

“Exogenous” refers to a molecule or substance (e.g., amino acid) that originates from outside a given cell or organism. Conversely, the term “endogenous” refers to a molecule or substance that is native to, or originates within, a given cell or organism.

“Isolated” or “purified” when referring to MIM-Cells means chemically induced neurons at least 30%, 35%, 40%, 45%, 50%, 55%, 60%, 65%, 70%, 75%, 80%, 85%, 90%, 95%, or 99% free of contaminating cell types such as non-neuronal cells. The isolated MIM-Cells may also be substantially free of soluble, naturally occurring molecules.

“Neuronal-like morphology” is used herein interchangeably with “neuron-morphology” to refer to morphological characteristic of neurons, such as the presence of a soma/cell body, dendrites, axon and/or synapses.

“Treating”, and/or “ameliorating” neurodegenerative or neurological disorders or neuronal injuries as used herein refer to reducing/decreasing the symptoms associated with the neurodegenerative or neurological disorders or neuronal injury.

### II. Compositions

Compositions include metabolites and metabolite cocktails for use in inducing cells into a change from their steady state. “Steady-state” refers to the time in which one cell maintains same identity (i.e. with metabolism and transcriptional programs that are the signature of that cell type). In this sense, examples of steady-states are the pluripotent stem cells, multipotent stem cells, and every cellular subtype differentiated from each lineage. In one embodiment, the metabolites and metabolite cocktails are used to induce cells into a less differentiated state, when compared to their original state before treatment. In other embodiments, the metabolites and metabolite cocktails are used to induce differentiation of less differentiated cells, for example, pluripotent and multipotent cells. The metabolites are used to supplement cell culture media, and accordingly, cells culture media supplemented with the disclosed metabolites are also provided.

The examples below demonstrate that C1-metabolites repress the gene expression phenotype of differentiated cells, as demonstrated by decreases in the expression of mature cell markers.

The disclosed compositions also include chemically reprogramed cells, which are obtained following culture in the metabolite supplemented cell culture media, supplemented with effective amount of the metabolite to reprogram the cells by reversing their state of differentiation into a less differentiated state.

A. Metabolites for Inducing De-Differentiation of Differentiated Cells

Metabolites are disclosed for use in inducing a wave of C1-metabolites in a cell, to de-differentiate the cell into a progenitor-like state. Treatment of fully differentiated cells with these metabolites caused the loss of cellular identity and transition toward progenitor-like states. The small molecules include methionine, SAM (S-Adenosyl methionine), s-adenosylhomocysteine (SAH), threonine, 2-amino-3-oxobutanoate, serine, ophtalmate, glutamate, 5-oxoproline, cysteineglycine, glycine, betaine, dimethylglycine, putrescine, spermidine, spermine, N-acetylspermine and N-acetylspermidine, and cysteine, cysteine sulfinic acid, hypotaurine, taurine, cystine, thiocystine, cysteine-glutathione disulfide, gamma-glutamyl-cysteine, S-methylglutathione, S-lactoylglutathione, glutathione reduced (GSG), glutathione oxidized (GSSG), N-acetylmethionine, N-acetylcysteine, methionine sulfone, N-acetyltaurine, N-formylmethionine, cysteine S-sulfate, methionine sulfoxide, N-acetylmethionine sulfoxide, S-methylcysteine, sulfo-L-alanine, N-acetylserine, phosphoserine, homoserinelactone, phosphothreonine, and N-acetylthreonine, 4-acetamidobutanoeate, N-acetyl isoputreanine, N-diacetylspermine, and N-acetylputrescine, 2-hydroxybutyrate, 3-dephospho-CoA-glutathione, dicarboxyethylglutathione, CoA-glutathione, 4-hydroxy-nonenal-glutathione, cyclic-dGSH, and S-nitrosoglutathione (GSNO) (herein, collectively, C1-metabolites). C1 metabolites are used alone or in combination (i.e., as a cocktail, herein, C1-Metabolic Induction Medium (C1-MIM)) to supplement basal cell culture media in effective amounts to induce C1-metabolism in a cell in cell culture media supplemented with the C1-metabolites or C1-MIM)).

B. MIM-Supplemented Media

Also provided is cell culture media, supplemented with MIM (MIM-supplemented media), disclosed herein. The MIM-supplemented media is obtained by introducing exogenous C1 metabolites into basal cell culture media, for example, used to culture differentiated cells, which are commercially available. Examples of commercially available base media may include, but are not limited to, phosphate buffered saline (PBS), Dulbecco's Modified Eagle's Medium (DMEM), Minimal Essential Medium (MEM), Basal Medium Eagle (BME), Roswell Park Memorial Institute Medium (RPMI) 1640, MCDB 131, Clicks medium, McCoy's 5 A Medium, Medium 199, William's Medium E, insect media such as Grace's medium, Ham's Nutrient mixture F-10 (Ham's F-10), Ham's F-12, a-Minimal Essential Medium (aMEM), Glasgow's Minimal Essential Medium (G-MEM), Iscove's Modified Dulbecco's Medium, Neurobasal media, DMEMF12 and MEM Alpha. One of ordinary skill in the art can readily select the cell culture medium to be supplemented, based on the cell-type.

In some embodiments, the basal cell culture medium is additionally supplemented with serum. In a preferred embodiment, however, the basal cell culture medium is serum free and is further supplemented with a cell survival factor such as fibroblast growth factor 2 (Fgf2), neutrophin, glial cell line-derived neurotrophic factor. etc, to maintain cell survival.

The basal cell medium is supplemented with at least two Cl metabolites, and in preferred embodiments, with a C1-MIM. The C1-MIM includes at least 3 C1 metabolites, 4 C1 metabolites, 5 C1 metabolites, for example, methionine, threonine, glycine, putrescine, SAM or methionine, threonine, glycine, putrescine, cysteine, or 6 C1 metabolites (i.e., methionine, threonine, glycine, putrescine, SAM and cysteine, MIM(6))). In a more preferable embodiment, 4 C1 metabolites are used, more preferably, methionine, threonine, glycine, putrescine (MIM(4)). The concentration of SAM preferably should not exceed 2 mM, preferably, it should not exceed 1.5 mM, and more preferably, it should not exceed 0.5 mM. Thus, SAM is preferably added to basal cell culture medium at a concentration ranging from 0.01 to 2mM, more preferably, between 0.1 and 1.5 mM and most preferably, between 0.1 and 0.5 mM.

Methionine is used to supplement basal cell culture medium at a concentration ranging from 0.001 to 100 mM, preferably between 0.025 and 50 MM, and most preferably, between 0.025 and 10 mM.

Glycine is used to supplement basal cell culture medium at a concentration ranging from 0.001 to 100 mM, preferably between 0.025 and 50 mM, and most preferably, between 0.025 and 10 mM.

Threonine is used to supplement basal cell culture medium at a concentration ranging from 0.001 to 100 mM, preferably between 0.025 and 50 mM, and most preferably, between 0.025 and 10 mM.

Putrescine is used to supplement basal cell culture medium at a concentration ranging from 0.001 to 100 mM, preferably between 0.025 and 50 mM, and most preferably, between 0.025 and 10 mM.

Cysteine is used to supplement basal cell culture medium at a concentration ranging from 0.001 to 100 mM, preferably between 0.025 and 50 mM, and most preferably, between 0.025 and 10 mM.

A particularly preferred C1-MIM used to supplement basal cell culture medium includes MIM(6) where 5 C1 metabolites (Gly, Thr, Cys, Putrescine and Met) are added to basal cell culture medium (without/without serum+FGF2) at a concentration of about 2.5 to about 5mM, preferably, 5mM with exception of SAM which is added at about 0.5mM, or MIM(4), where 5 Cl metabolites (Gly, Thr, Cys, Putrescine and Met) are added to basal cell culture medium (without/without serum+FGF2) at a concentration of about 2.5 to about 5mM, preferably, 5mM, with exception of SAM which is added at about 0.5mM.

C. Cells to be Induced and MIM-Induced Cells

MIM-Cells are obtained by inducing cells obtained from any mammal, for example, partially or completely differentiated cells obtained from any mammal (e.g., bovine, ovine, porcine, canine, feline, equine, primate), preferably a human. Sources include bone marrow, fibroblasts, fetal tissue (e.g., fetal liver tissue), peripheral blood, umbilical cord blood, pancreas, skin or any organ or tissue. However, MIM-Cells can be obtained from other cell types including, but not limited to: stem cells, multipotent stem cell types, myoblasts (MBs), neural stem cells (NSCs), mesenchymal stem cells (MSCs)), cells of hematological origin, cells of embryonic origin, skin derived cells, adipose cells, epithelial cells, endothelial cells, mesenchymal cells, parenchymal cells, neurological cells, and connective tissue cells. In a preferred embodiment, the MIM-Cells are obtained from chemically induced fibroblasts, chondrocytes, neurons, and astrocytes.

The cell to be re-programmed in its fate can be obtained from a sample obtained from a mammalian subject. The subject can be any mammal (e.g., bovine, ovine, porcine, canine, feline, equine, primate), including a human. A sample of cells may be obtained from any of a number of different sources including, for example, bone marrow, fetal tissue (e.g., fetal liver tissue), peripheral blood, umbilical cord blood, pancreas (beta cells, are alpha, delta, gamma, and epsilon cells islet cells, gamma cells)), skin or any organ or tissue.

Cells may be isolated by disaggregating an appropriate organ or tissue which is to serve as the cell source using techniques known to those skilled in the art. For example, the tissue or organ can be disaggregated mechanically and/or treated with digestive enzymes and/or chelating agents that weaken the connections between neighboring cells, so that the tissue can be dispersed to form a suspension of individual cells without appreciable cell breakage. Enzymatic dissociation can be accomplished by mincing the tissue and treating the minced tissue with one or more enzymes such as trypsin, chymotrypsin, collagenase, elastase, and/or hyaluronidase, DNase, pronase, dispase etc. Mechanical disruption can also be accomplished by a number of methods including, but not limited to, the use of grinders, blenders, sieves, homogenizers, pressure cells, or insonators.

MIM cells differ from the cells from which they were obtained (herein, the parent cell) in that the show decreases in the expression of at least one mature cell marker when compared to the parent cell; associated by changes in the cell morphology, when comparing the MIM-Cell to the parent cell. In a preferred embodiment, the MIM cells are not genetically engineered, i.e., the MIM cells are not altered by introducing or removing genetic elements from the cells.

Mature cell markers are used herein to refer to markers used to identify committed cells, and such markers are known in the art. Not limiting examples are disclosed herein. Neuron specific markers include TUJ1 (Neuron-specific class III beta-tubulin), MAP2, NF-H and NeuN. MAP-2 is a neuron-specific cytoskeletal protein that is used as a marker of neuronal phenotype. Izant, et al., Proc Natl Acad Sci U S A., 77:4741-5 (1980). NeuN is a neuronal specific nuclear protein identified by Mullen, et al., Development, 116:201-11 (1992). Fibroblast hallmark genes include Fap, Des, Slug, Dcn, FSp1, Tgfb1i1, Snail, Collagen 1 and Twist2. Hepatocyte cell markers include, but are not limited to albumin, Cytochrome P450 (Cyp)3A4, CYPB6, CYP1A2, CYP2C9, and/or CYP2C19; adipocyte markers include for example, adiponectin, fatty acid binding protein P4, and leptin.

For example an MIM-cells obtained from astrocytes show decreased expression of the astrocytic markers such as Glial fibrillary acidic protein encoding gene, Gfap and Cd44 (Cluster of differentiation 44); MIM-cells obtained from chondrocytes show decreased expression of the chondrocyte markers such as Aggrecan, collagen type II (Co/2); MIM-cells obtained from neurons show decreased expression of neuronal markers such as Map2 and beta-III-tubulin; and MIM-cells obtained from fibroblast show decreased expression of fibroblast markers, such as collagen type I alpha 2 chain encoding gene, Col1A2. The decrease in the expression of at least one mature cell marker when compared to the parent cell can be determined using methods known in the art The MIM-Cells differ from the parent cell in some embodiments in up 10% decreases in the expression of at least one mature cell marker, at least a 20% decrease, a 40% decrease, a 50% decrease, at least a 60%, 70%, or 80% decrease and in some embodiments, up to 95% decrease in the expression of at least one mature cell marker. Also, MIM-Cells express genes associated with progenitor states, for example, cMyc, Ascll, SOX2, Nestin, CD133 and Pax7, and in some embodiments, MIM-Cell do not express Oct4.

D. MIM-Cell-Based Therapeutic Compositions

MIM-Cells can be formulated for administration, delivery or contacting a subject, tissue or cell using a suitable pharmaceutically acceptable carrier. In some embodiments, the cells are simply suspended in a physiological buffer. In other embodiments, the cells are provided with, or incorporated into a support structure. One strategy includes encapsulating/suspending MIM-Cells in a suitable polymeric support. The support structures may be biodegradable or non-biodegradable, in whole or in part. The support may be formed of a natural or synthetic polymer. Natural polymers include collagen, hyaluronic acid, polysaccharides, alginates and glycosaminoglycans. Synthetic polymers include polyhydroxyacids such as polylactic acid, polyglycolic acid, and copolymers thereof, polyhydroxyalkanoates such as polyhydroxybutyrate, polyorthoesters, polyanhydrides, polyurethanes, polycarbonates, and polyesters. These may be in for the form of implants, tubes, meshes, or hydrogels. The support structure may be a loose woven or non-woven mesh, where the cells are seeded in and onto the mesh. The structure may include solid structural supports. The support may be a tube, for example, a neural tube for regrowth of neural axons.

For example, the cells can be suspended in a hydrogel matrix of collagen, alginate or Matrigel®. Common non-biodegradable cell-carriers in neural tissue engineering include silicone, polyvinylv alcohol (PVA) and copolymer poly(acrylonitrile-co-vinyl chloride) (P(AN/VC)), polysulphone (PSU) and poly(ethersulphone) (PES), poly(ethylene terephthalate) (PET) and polypropylene (PP). Reviewed in Wong, et al., Int. J. Mol Sci. 15:10669-10723 (2014). See also, Blurton-Jones, et al., Proc. Natl. Acad.

Sci., 106 (32):13594-9 (2009); Jin, et al, J. Cereb. Blood Flow Metab., 30:534-44 (2009); and Lundberg, et al, Neuroradiology, 51:661-7 (2009). Transplantation of microencapsulated cells is known in the art, and is disclosed for example in Balladur et al., Surgery, 117:189-94 (1995); and Dixit et al., Cell Transplantation, 1:275-79 (1992). Additional therapeutic compositions including MIM-Cells are described below under method of using.

The MIM-Cell-based therapeutic compositions include effective amounts of MIM-Cells for use in the methods disclosed herein. For example, a dose of 10⁴-10⁵ cells can be initially administered, and the subject monitored for an effect (e.g., engraftment of the cells, improved neural function, increased neuronal density in an affected area). The dose MIM-Cells can be in the range of 10³-10⁷, 10⁴-10′⁷, 10⁵-10⁸, 10⁶-10⁹, or 10⁶-10⁸ cells. The pharmaceutical preparation including MIM-Cells can be packaged or prepared in unit dosage form. The cells can be lyophilized and/ or frozen for increased shelf life, and resuspended prior to administration. In such form, the cellular preparation is subdivided into unit doses containing appropriate quantities of the active component, e.g., according to the dose of the therapeutic agent. The unit dosage form can be a packaged preparation, the package containing discrete quantities of preparation. The composition can, if desired, also contain other compatible therapeutic agents.

### III. Methods of Making

A. Preparation of MIM-Cells

MIM-Cells can be prepared following the protocol outlined in the examples below and described briefly here. Cells to be induced (steady state cells) are isolated as disclosed herein and cultured in suitable primary cell culture media (based on the tissue source of the isolated cells. Cell cultures seeded in adherent conditions are kept on their respective differentiation media until reaching a mature phenotype. Mature phenotype, here is understood as the cell type obtained after a cell type is maintained in differentiation medium (usually an standard known for each cell type) reaches the expression of markers (at RNA or protein level) which identify the cell as terminal differentiated. For example, Map2 for neurons. When cultures reach about 80-90% confluence, the differentiation media is removed, cells were washed with PBS and the media replaced with MIM-supplemented media (described above). The MIM-supplemented medium is preferably serum free or contains reduced serum (about 2%) in some embodiments, and includes the growth factor FGF2 (20 ng/mL). MIM-Cells can be harvested following culture in MIM-supplemented cell culture media, for about one to 5 days, but this time could be adjusted (reduced or expanded) depending of the cell type to be treated.

The disclosed MIM-Cells are not obtained by transfecting the parent cell to express any of Oct4, KLF4, SOX2, C-Myc or NANOG.

B. Culture and Preservation of MIM-Cells

The MIM-cells can be expanded in culture and stored for later retrieval and use. Once a culture of cells is established, the population of cells is mitotically expanded in vitro by passage to fresh medium as cell density dictates, under conditions conducive to cell proliferation, with or without tissue formation. Such culturing methods can include, for example, passaging the cells in culture medium lacking particular growth factors that induce differentiation (e.g., IGF, EGF, FGF, VEGF, and/or other growth factor). Cultured cells can be transferred to fresh medium when sufficient cell density is reached. Cell culture medium for maintaining neuronal cells are commercially available.

Cells can be cryopreserved for storage according to known methods, such as those described in Doyle, et al., (eds.), 1995, Cell & Tissue Culture: Laboratory Procedures, John Wiley & Sons, Chichester. For example, cells may be suspended in a “freeze medium” such as culture medium containing 15-20% fetal bovine serum (FBS) and 10% dimethylsulfoxide (DMSO), with or without 5-10% glycerol, at a density, for example, of about 4-10 x 10⁶ cells/ml. The cells are dispensed into glass or plastic vials which are then sealed and transferred to a freezing chamber of a programmable or passive freezer. The optimal rate of freezing may be determined empirically. For example, a freezing program that gives a change in temperature of -1 ° C/min through the heat of fusion may be used. Once vials containing the cells have reached -80 ° C., they are transferred to a liquid nitrogen storage area. Cryopreserved cells can be stored for a period of years.

### IV. Methods of Use

Identification of a readily available source of progenitor-like cells that can give rise to a desired cell type or morphology is important for therapeutic treatments, tissue engineering and research. The availability of progenitor-like cells would be extremely useful in transplantation, tissue engineering, regulation of angiogenesis, vasculogenesis, and cell replacement or cell therapies as well as the prevention of certain diseases. Such cells can also be used to introduce a gene into a subject as part of a gene therapy regimen.

A. Cell Therapy

Therapeutic uses of the MIM-cells include transplanting the induced MIM-cells, or progeny thereof into individuals to treat a variety of pathological states including diseases and disorders resulting from cancers, wounds, neoplasms, injury, viral infections, diabetes and the like. Treatment may entail the use of the cells to produce new tissue, and the use of the tissue thus produced, according to any method presently known in the art or to be developed in the future. The cells may be implanted, injected or otherwise administered directly to the site of tissue damage so that they will produce new tissue in vivo.

De-differentiated Neuronal Cells

MIM-cells obtained from differentiated cells of neuronal origin (herein, MIM-neuronal cells) are useful in methods for treating and/or ameliorating neurodegenerative or neurological disorders or neuronal injuries in a subject in need thereof (individuals having a neuronal cell deficiency). In a preferred embodiment, the MIM-neuronal cells are obtained from autologous cells, i.e., the donor cells are autologous. However, the cells can be obtained from heterologous cells. In one embodiment, the donor cells are obtained from a donor genetically related to the recipient. In another embodiment, donor cells are obtained from a donor genetically un-related to the recipient. If the human MIM-neuronal cells are derived from a heterologous (non-autologous/allogenic) source compared to the recipient subject, concomitant immunosuppression therapy is typically administered, e.g., administration of the immunosuppressive agent cyclosporine or FK506.

The method includes administering to the individual/subject an effective amount of MIM-neuronal cells, thereby treating and/or ameliorating symptoms associated with the neurodegenerative disorder or neuronal injury. In some embodiments, the MIM-neuronal cells are administered to the site of the neurodegeneration or neuronal injury in the individual for example, by injection into the lesion site using a syringe positioning device. MIM-neuronal cells can be transplanted directly into parenchymal or intrathecal sites of the central nervous system, according to the disease being treated (U.S. Pat . No. 5,968,829 for example). MIM-neuronal cells can be administered to a subject in need thereof, using known methods of administering cells to neuronal tissues such as the brain or spinal as described for example in Blurton-Jones, et al., Proc. Natl. Acad. Sci., 106 (32):13594-9 (2009); Jin, et al, J. Cereb. Blood Flow Metab., 30:534-44 (2009); and Lundberg, et al, Neuroradiology, 51:661-7 (2009).

As with any therapy, the course of treatment is best determined on an individual basis depending on the particular characteristics of the subject and the type of treatment selected. The treatment can be administered to the subject one time, on a periodic basis (e.g., bi-weekly, monthly) or any applicable basis that is therapeutically effective. The treatment can be administered alone or in combination with another therapeutic agent, e.g., an agent that reduces pain, or an agent that encourages neuronal function or growth. The additional therapeutic agent can be administered simultaneously with the MIM-neuronal cells, at a different time, or on an entirely different therapeutic schedule (e.g., the MIM-neuronal cells can be administered as needed, while the additional therapeutic agent is administered daily or weekly).

The dosage of MIM-neuronal cells administered to a patient will vary depending on a wide range of factors. For example, it would be necessary to provide substantially larger doses to humans than to smaller animals. The dosage will depend upon the size, age, sex, weight, medical history and condition of the patient, use of other therapies, and the frequency of administration. However, those of ordinary skill in the art can readily determine appropriate dosing, e.g., by initial animal testing, or by administering relatively small amounts and monitoring the patient for therapeutic effect. If necessary, incremental increases in the dose can be administered until the desired results are obtained. Generally, treatment is initiated with smaller dosages which may be less than the optimum dose of the therapeutic agent. Thereafter, the dosage is increased by small increments until the optimum effect under circumstances is reached.

Individuals or subjects in need of the MIM-neuronal cells disclosed herein include, but are not limited to, subjects with a neurodegenerative disorder selected from the group consisting of Alzheimer's Disease (AD), Huntington's Disease (HD), Parkinson's Disease (PD) Amyotrophic Lateral Sclerosis (ALS), Multiple Sclerosis (MS) and Cerebral Palsy (CP),

Dentatorubro-pallidoluysian Atrophy (DRPLA), Neuronal Intranuclear Hyaline Inclusion Disease (NIHID), dementia with Lewy bodies, Down's Syndrome, Hallervorden-Spatz disease, prion diseases, argyrophilic grain dementia, cortocobasal degeneration, dementia pugilistica, diffuse neurofibrillary tangles, Gerstmann-Straussler-Scheinker disease, Hallervorden-Spatz disease, Jakob-Creutzfeldt disease, Niemann-Pick disease type 3, progressive supranuclear palsy, subacute sclerosing panencephalitis, Spinocerebellar Ataxias, Picks disease, and dentatorubral-pallidoluysian atrophy. Neuronal injury includes, but is not limited to traumatic brain injury, stroke, and chemically induced brain injury. Neuronal injuries can result from any number of traumatic incidents, e.g., obtained in sport, accident, or combat. Neuronal injuries include concussion, ischemia (stroke), hemorrhage, or contusion resulting in damage to the neurons in an individual or significant loss of neuronal tissue in drastic cases. Also included are neuronal injuries and loss caused by pathogenic infection, or chemically induced brain injury, e.g., due to medication, environmental factors, or substance abuse.

Methods for diagnosing neurodegenerative disorders, neurological disorders, and neuronal injuries are known in the art (see, e.g., Diagnostic and Statistical Manual of Mental Disorders, Fourth Edition (DSM-IV-TR), American Psychiatric Assoc. 2000). Generally, a physician or neurologist will consider a number of factors in making a diagnosis in a particular individual or patient. For example, family history is often indicative of a risk of AD, HD, PD, and other neurodegenerative disorders. Doctors will also carry out chemical tests to check for normal blood count, thyroid function, liver function, glucose levels. Spinal fluid is often analyzed as part of this testing. Neuropsychological tests can also be used to assess memory, problem-solving, decision making, attention, vision-motor coordination and abstract thinking. These include spatial exercises and simple calculations.

The Mini-Mental State Examination is also common. CAT scans and MRIs can also be used to rule out tumors, and can provide clues as to degraded areas of the brain. Non-invasive medical imaging techniques such as Positron Emisson Tomography (PET) or single photon emission computerized tomography (SPECT) imaging are particularly useful for the detection of brain disease. PET and SPECT imaging shows the chemical functioning of organs and tissues, while other imaging techniques, such as X-ray, CT and MRJ, show structure. The use of PET and SPECT imaging has become increasingly useful for qualifying and monitoring the development of brain diseases. In some instances, the use of PET or SPECT imaging allows a neurodegenerative disorder to be detected several years earlier than the onset of symptoms. Once an individual has been diagnosed as having a deficiency in neuronal cells, e.g., resulting from neurodegeneration or injury, the individual can be considered for treatment with the cell-based therapies described herein.

Diabetes

Diabetes mellitus (DM) is a group of metabolic diseases where the subject has high blood sugar, either because the pancreas does not produce enough insulin, or, because cells do not respond to insulin that is produced.

A promising replacement for insulin therapy is provision of islet cells to the patient in need of insulin. Shapiro et al., N Engl J Med., 343(4):230-8 (2000) have demonstrated that transplantation of beta cells/islets provides therapy for patients with diabetes. Although numerous insulin types are commercially available, these formulations are provided as injectables. The conversion of differentiated nonbeta cell types in the pancreas into beta cells through transdifferentiation has the potential to restore glucose homeostasis. Importantly, nonbeta cells are still present in normal or even increased numbers in the diabetic pancreas, and therefore represent a potential source of replacement beta cells. MIM-cells of pancreatic origin can provide an alternative source of islet cells to prevent or treat diabetes. For example, MINI-cells can be isolated and differentiated to a pancreatic cell type and delivered to a subject. Alternatively, the MIM-cells can be delivered to the pancreas of the subject and differentiated to islet cells in vivo. Accordingly, the cells are useful for transplantation in order to prevent or treat the occurrence of diabetes. Methods for reducing inflammation after cytokine exposure without affecting the viability and potency of pancreatic islet cells are disclosed for example in U.S. Pat. No. 8,637,494 to Naziruddin, et al.

Tissue Engineering

MIM-Cells and their progeny can be used to make tissue engineered constructions, using methods known in the art. Tissue engineered constructs may be used for a variety of purposes including as prosthetic devices for the repair or replacement of damaged organs or tissues. They may also serve as in vivo delivery systems for proteins or other molecules secreted by the cells of the construct or as drug delivery systems in general. Tissue engineered constructs also find use as in vitro models of tissue function or as models for testing the effects of various treatments or pharmaceuticals. The most commonly used biomaterial scaffolds for transplantation of stem cells are reviewed in the most commonly used biomaterial scaffolds for transplantation of stem cells is reviewed in Willerth, S.M. and Sakiyama-Elbert, S.E., Combining stem cells and biomaterial scaffolds for constructing tissues and cell delivery (July 9, 2008), StemBook, ed. The Stem Cell Research Community, StemBook. Tissue engineering technology frequently involves selection of an appropriate culture substrate to sustain and promote tissue growth. In general, these substrates should be three-dimensional and should be processable to form scaffolds of a desired shape for the tissue of interest.

U.S. Pat. No. 6,962,814 generally discloses method for producing tissue engineered constructs and engineered native tissue. With respect to specific examples, U.S. Pat. No. 7,914,579 to Vacanti, et al., discloses tissue engineered ligaments and tendons. U.S. Pat. No. 5,716,404 discloses methods and compositions for reconstruction or augmentation of breast tissue using dissociated muscle cells implanted in combination with a polymeric matrix. U.S. Pat. No. 8,728,495 discloses repair of cartilage using autologous dermal fibroblasts. U.S. Published application No. 20090029322 by Duailibi, et al., discloses the use of stem cells to form dental tissue for use in making tooth substitute. U.S. Published application No. 2006/0019326 discloses cell-seed tissue-engineered polymers for treatment of intracranial aneurysms. U.S. Published application No. 2007/0059293 by Atala discloses the tissue-engineered constructs (and method for making such constructs) that can be used to replace damaged organs for example kidney, heart, liver, spleen, pancreas, bladder, ureter and urethra.

Therapeutic Compositions

A combination of C1-metabolites as discloses herein or the MIM-Cells can be formulated for administration, delivery or contacting with a subject, tissue or cell to promote modulation of cellular steady state, for example, de-differentiation in vivo or in vitrolex vivo. Additional factors, such as growth factors, other factors that induce differentiation or dedifferentiation, secretion products, immunomodulators, anti-inflammatory agents, regression factors, biologically active compounds that promote innervation, vascularization or enhance the lymphatic network, and drugs, can be incorporated.

i. C1-metabolite Compositions

The C1-metabolites can be administered to a subject in need thereof, in effective amounts to modulate cellular steady state in the subject. The metabolites can be administered in a pharmaceutically acceptable carrier, or used to supplement a diet. Suitable oral dosage forms include tablets, capsules, solutions, suspensions, syrups, and lozenges. Tablets can be made using compression or molding techniques well known in the art. Gelatin or non-gelatin capsules can prepared as hard or soft capsule shells, which can encapsulate liquid, solid, and semi-solid fill materials, using techniques well known in the art.

One or more C1compounds, and optional one or more additional active agents, can be incorporated into microparticles, nanoparticles, or combinations thereof, that provide release of the compound(s) e.g., delayed, extended, immediate, or pulsatile). Release of the compounds is controlled by diffusion of the drug(s) out of the microparticles and/or degradation of the polymeric particles by hydrolysis and/or enzymatic degradation. Suitable polymers include ethylcellulose and other natural or synthetic cellulose derivatives. Suitable polymers include ethylcellulose and other natural or synthetic cellulose derivatives.

Polymers, which are slowly soluble and form a gel in an aqueous environment, such as hydroxypropyl methylcellulose or polyethylene oxide, can also be suitable as materials for drug containing microparticles. Other polymers include, but are not limited to, polyanhydrides, poly(ester anhydrides), polyhydroxy acids, such as polylactide (PLA), polyglycolide (PGA), poly(lactide-co-glycolide) (PLGA), poly-3-hydroxybutyrate (PHB) and copolymers thereof, poly-4-hydroxybutyrate (P4HB) and copolymers thereof, polycaprolactone and copolymers thereof, and combinations thereof. The nano and microparticles including one or more Cl compounds can be prepared using methods known in the art. Encapsulation or incorporation of drug into carrier materials to produce drug-containing microparticles can be achieved through known pharmaceutical formulation techniques. In the case of formulation in fats, waxes or wax-like materials, the carrier material is typically heated above its melting temperature and the drug is added to form a mixture comprising compound particles suspended in the carrier material, compound dissolved in the carrier material, or a mixture thereof. Microparticles can be subsequently formulated through several methods including, but not limited to, the processes of congealing, extrusion, spray chilling or aqueous dispersion. In a preferred process, wax is heated above its melting temperature, compound is added, and the molten wax-compound mixture is congealed under constant stirring as the mixture cools. Alternatively, the molten wax-compound mixture can be extruded and spheronized to form pellets or beads. These processes are known in the art.

For some carrier materials it may be desirable to use a solvent evaporation technique to produce compound-containing microparticles. In this case compound and carrier material are co-dissolved in a mutual solvent and microparticles can subsequently be produced by several techniques including, but not limited to, forming an emulsion in water or other appropriate media, spray drying or by evaporating off the solvent from the bulk solution and milling the resulting material.

In some embodiments, compounds in a particulate form is homogeneously dispersed in a water-insoluble or slowly water-soluble material. To minimize the size of the compound particles within the composition, the compound powder itself may be milled to generate fine particles prior to formulation. The process of jet milling, known in the pharmaceutical art, can be used for this purpose. In some embodiments compound in a particulate form is homogeneously dispersed in a wax or wax like substance by heating the wax or wax like substance above its melting point and adding the compound particles while stirring the mixture. In this case a pharmaceutically acceptable surfactant may be added to the mixture to facilitate the dispersion of the active agent (herein Cl compounds) particles.

ii. MIM-Cell-based Compositions

The MIM-Cells can be administered to a patient by way of a composition that includes a population of MIM-Cells or MIM-Cells progeny alone or on or in a carrier or support structure. In many embodiments, no carrier will be required. The cells can be administered by injection onto or into the site where the cells are required. In these cases, the cells will typically have been washed to remove cell culture media and will be suspended in a physiological buffer.

In other embodiments, the cells are provided with or incorporated onto or into a support structure. Support structures may be meshes, solid supports, scaffolds, tubes, porous structures, and/or a hydrogel. The support structures may be biodegradable or non-biodegradable, in whole or in part. The support may be formed of a natural or synthetic polymer, metal such as titanium, bone or hydroxyapatite, or a ceramic. Natural polymers include collagen, hyaluronic acid, polysaccharides, and glycosaminoglycans. Synthetic polymers include polyhydroxyacids such as polylactic acid, polyglycolic acid, and copolymers thereof, polyhydroxyalkanoates such as polyhydroxybutyrate, polyorthoesters, polyanhydrides, polyurethanes, polycarbonates, and polyesters. These may be in for the form of implants, tubes, meshes, or hydrogels.

Solid Supports

The support structure may be a loose woven or non-woven mesh, where the cells are seeded in and onto the mesh. The structure may include solid structural supports. The support may be a tube, for example, a neural tube for regrowth of neural axons. The support may be a stent or valve. The support may be a joint prosthetic such as a knee or hip, or part thereof, that has a porous interface allowing ingrowth of cells and/or seeding of cells into the porous structure. Many other types of support structures are also possible. For example, the support structure can be formed from sponges, foams, corals, or biocompatible inorganic structures having internal pores, or mesh sheets of interwoven polymer fibers. These support structures can be prepared using known methods.

The support structure may be a permeable structure having pore-like cavities or interstices that shape and support the hydrogel-cell mixture. For example, the support structure can be a porous polymer mesh, a natural or synthetic sponge, or a support structure formed of metal or a material such as bone or hydroxyapatite. The porosity of the support structure should be such that nutrients can diffuse into the structure, thereby effectively reaching the cells inside, and waste products produced by the cells can diffuse out of the structure.

The support structure can be shaped to conform to the space in which new tissue is desired. For example, the support structure can be shaped to conform to the shape of an area of the skin that has been burned or the portion of cartilage or bone that has been lost. Depending on the material from which it is made, the support structure can be shaped by cutting, molding, casting, or any other method that produces a desired shape. The support can be shaped either before or after the support structure is seeded with cells or is filled with a hydrogel-cell mixture, as described below.

An example of a suitable polymer is polyglactin, which is a 90:10 copolymer of glycolide and lactide, and is manufactured as VICRYL™ braided absorbable suture (Ethicon Co., Somerville, N.J.). Polymer fibers (such as VICRYL™), can be woven or compressed into a felt-like polymer sheet, which can then be cut into any desired shape. Alternatively, the polymer fibers can be compressed together in a mold that casts them into the shape desired for the support structure. In some cases, additional polymer can be added to the polymer fibers as they are molded to revise or impart additional structure to the fiber mesh. For example, a polylactic acid solution can be added to this sheet of polyglycolic fiber mesh, and the combination can be molded together to form a porous support structure. The polylactic acid binds the cros slinks of the polyglycolic acid fibers, thereby coating these individual fibers and fixing the shape of the molded fibers. The polylactic acid also fills in the spaces between the fibers. Thus, porosity can be varied according to the amount of polylactic acid introduced into the support. The pressure required to mold the fiber mesh into a desirable shape can be quite moderate. All that is required is that the fibers are held in place long enough for the binding and coating action of polylactic acid to take effect.

Alternatively, or in addition, the support structure can include other types of polymer fibers or polymer structures produced by techniques known in the art. For example, thin polymer films can be obtained by evaporating solvent from a polymer solution. These films can be cast into a desired shaped if the polymer solution is evaporated from a mold having the relief pattern of the desired shape. Polymer gels can also be molded into thin, permeable polymer structures using compression molding techniques known in the art.

Hydrogels

In another embodiment, the cells are mixed with a hydrogel to form a cell-hydrogel mixture. Hydrogels may be administered by injection or catheter, or at the time of implantation of other support structures. Crosslinking may occur prior to, during, or after administration.

### V. Kits

Kits are provided which include C1-metabolites and/or C1-MIM disclosed herein. The C1-metabolites and/or C1-MIM are as described above. These may be in a form having defined concentrations to facilitate addition to cell culture media to produce a desired concentration. The kit may include directions providing desired concentration ranges and times of administration based on the types of cells to be induced. The kit may also include cell culture media pre-mixed with the C1-metabolites and/or C1-MIM for culture of terminally or partially differentiated cells to induce de-differentiation into a less differentiated stated and a progenitor-like state, characterized in a reduction of at least one at least one mature cell marker and an upregulation in the expression of at least one genes characteristic of a progenitor state.

The present invention will be further understood by reference to the following non-limiting examples.

### EXAMPLE

### Example 1

### Metabolomic Transitions Between Two Cellular identities

Materials and Methods

Animals

ICR mice were purchased from the Jackson laboratory. All mouse experiments were approved by the IACUC committee to conform to regulatory standards.

Reagents

From Thermo Fisher Scientific: Neurobasal media (Cat. 21103-049), DMEMF12 (Cat. 11330-032), DMEM (Cat. 11995-040), MEM Alpha (Cat. 12571-048), B27 (Cat. 17504-044), N2 (Cat. 17502-048), MEM-Non-Essential Amino Acids Solution (Cat. 11140050), rhEFG, (Cat. PH G0311),

Fetal Bovine Serum (Cat. 16000-044), GlutaMAX (Cat. 35050-061), STEMPro Chondrogenesis Differentiation Kit (Cat. A10069), Pen-Strep (Cat. 15140-122), TrypLE (Cat. 12604021), β-mercaptoethanol (Cat. 31350010), Trypsin Inhibitor (Cat. 17075029), Maxima H Minus cDNA Synthesis Master MIX (Cat. M1662), LDS sample buffer (Cat. 84788), Lipofectamine 2000 (Cat. 11668019), F-10 Ham's medium (Cat. 11550043).

From FISHER Bioreagents: Bovine Serum Albumin (Cat. 9048-46-8), Tween 20 (Cat. BP337-500).

From Ambion by Life technologies: TRIZOL (Cat. 15596018). From Innovative Cell Technologies: Accutase (Cat. AT104).

From ScienCell: Astrocyte Growth Supplement (Cat. 1852). From BD Bioscience: BD Matrigel Matrix (Cat. 354234). From Joint Protein Central: hFGF2 (Cat. BBI-EXP-002). From Reagents Direct: Y27632 (Cat. 53-B85-50).

From MILTENYI BIOTEC: LDN193189 (Cat. 130-106-540), Anti-GLAST (ACSA-1) Microbead Kit (Cat.130-095-825).

From Stem Cell Technologies: mTeSR™1 (Cat. 85850), Anti-Adherence Rinsing Solution (Cat. 07010).

From Tocris: SB 431542 (Cat. 1614). From BIORAD: SsoAdvanced Universal SYBR *Green Supermix (Cat. 1725274).

From QIAGEN: RNeasy Plus Mini kit (Cat. 74106).

From Sigma-Aldrich: Trypsin (Cat. T4674), Tritonx100 (Cat. T8787), DTT 50mM (Cat. Y00147), Poly-L-lysine (Cat. 150177), Collagenase/dispase (Roche, Cat. 10269638001), Insulin (Cat. 16634), L-Methionine (Cat. M5308), L-Threonine (Cat. T8441), Glycine (Cat. G5417), Putrescine dihydrochloride (Cat. P5780), L-cysteine (Cat. C7477), L-Arginine (Cat. A8094), Creatine (Cat. C0780), D-Fructose (Cat. F0127), L-Histidine (Cat. H5659), L-Leucine (Cat. L8912), L-Valine (Cat. V0513), Taurine (Cat. T8691). siRNAs Oligo-Names:
SASI_Mm02_00338586/MAT1A, SASI_Mm02_00338586AS/MAT1A,
SASI_Mm02_00295461/MTR, SASI_Mm02_00295461AS/MTR.

From Cayman Chemical: S-adenosylmethionine tosylate (Cat. 16376), cAMP (Cat. 18820).

From OZ Biosciences: Magnetic plate (Cat. MF10000), Combimag (Cat. CM20200).

From ABCAM: Methionine Assay Kit (Fluorometric) (Cat. ab234041).

From Cell Biolabs: S-Adenosylmethionine (SAM) ELISA Kit (Cat. STA-672).

From Vector: DAPI Vectashield (Cat. H 1200).

From Neuvitro: Poly-D-Lysine coverslips (12mm and 22mm) (Cat. H-12-1.5-PDL, GG-22-1.5-PDL).

Cell Cultures

Primary Culture of NSCs: NSCs were derived from murine embryonic cortex at 14.5 embryonic days (vaginal plug considered 0.5 days). A single-cell suspension was seeded in anti-adherent solution-treated dishes wish Neurobasal medium supplemented with 1X B27 and 20ng/mL of rh-EGF and h-FGF2. Primary neurospheres appeared after 5-6 days of culture and were used only during the first 10 passages. Cultures for NSCs were seeded at 200,000 cells/mL and maintained on standard conditions.

Astrocyte cultures:

Astrocytes were derived from cortical tissues postnatal or differentiated from dissociated neurospheres after a second or third passage, as described herein. For postnatal astrocytes, cortices were isolated from P4 mice pups to get single cells and were plated as described by Schildge et al.⁵¹Confluent cultures were sorted with Anti-GLAST (ACSA-1) Microbead kit according to manufacturer instructions. For inducing differentiation from NSCs, single cells derived from neurospheres were exposed to astrocyte media. In both cases, cells were plated over pre-treated Poly-L-Lysine plates or Poly-D-Lysine glass-coverslips using Astrocyte Differentiation Medium (DMEM-F12, 10% heat-inactivated FBS, lx B27, and 1X Glutamax). The media was replaced every other day until day-8 when 60 astrocytes reached a mature astrocytic phenotype corroborated by immunocytochemistry.

Neuronal cultures:

Primary neurons were obtained from the cortex of E14.5 mice brains. Brain dissection was performed in a cold solution of 2% glucose in PBS. Then, tissue was trypsinized, and the suspension was transferred across a 40 μm cell strainer to get a single cell suspension. Cells were plated in a ratio of 800,000 cells per each 22 mm poly-D-lysine coverslip with Neuron differentiation medium (Neurobasal media supplemented 1X B27 and 1X Glutamax). Cultures were maintained under standard conditions. The half volume of culture media was replaced every other day. In previous studies^{52,53}, tracked the disappearance of the proliferative neuronal progenitors present in the primary culture by 10 μM EdU-pulses every day after plating, and found that 5 days after seeding, the percentage of EdU³⁰ cells was reduced to basal levels. Neurons at this time point are considered as post-mitotic.

Primary cell cultures of adult brain tissues: Cerebella from 18.5 months old adult mice were dissociated, trypsinized, and the cell suspension derived therefrom was filtered across 70pm cell strainer. Cells were plated in 72 dishes coated with poly-L-lysine using Astrocyte Differentiation Medium, as described for astrocyte cultures. Cells were passaged when they reached the confluence of 90-100% in a 1:3 ratio. Cultures were used at passage 4 to 15. Specifically for re-differentiation experiments, cerebellar astrocytes were exposed to C1-MIM (see the correspondent section below) for 3-days, then switched into NSCs media for 1-day; finally, re differentiated by recovering the cells with TrypLE and re-seeding them on Poly-L-Lysine dishes (for downstream rtPCR analyses) or Poly-D-lysine coverslips (for immunocytochemical analyses), using a broad differentiation medium (Neurobasal, 1X B27, 1X N2, 1X Glutamax, 2.5mM Taurine, and 100 μM cAMP).

Glioblastoma Culture:

Tumor cells obtained from mouse glioblastoma multiforme—like tumors (mice ID 005) (Marumoto, et al. Nat. Med. 15, 110-116 (2009)) were used for these experiments. The cells were grown in DMEM supplemented with 1X N2, 20ng/mL rhEGF, 20ng/μL FGF2. Cultures were maintained in standard conditions.

Primary culture of chondrocytes:

Primary chondrocytes from femoral and tibial condyles of 5 days old mice were isolated as described by Gosset et al.,⁵⁵, and cultured overnight at a density of 7×10\hat{ }3 cells/cm². Chondrocyte medium contains DMEM plus 10% heat-inactivated FBS and 1X Glutamax. The media was replaced the following day with fresh media. Cultures were fed every other day.

MSC-cell line culture and chondrocyte differentiation:

MSCs were acquired from Cyagen (OriCell Strain C57BL/6 88 Mouse Adipose-Derived Mesenchymal Stem Cells) were thawed in StemXVivo® medium, and expanded using MSC Maintenance Medium (alpha-MEM plus 10% heat-inactivated FBS and 1X Glutamax). MSCs cells were differentiated into chondrocytes by using StemPro® Chondrogenesis Differentiation Kit by the 3D-culture system during the needed times (as indicated in the corresponding figure legends). Briefly, 2.5×10\hat{ }6 MSCs were resuspended in Chondrocyte differentiation medium and pelleted down in 15 mL polypropylene tubes, then, the caps were loosened, and the tubes placed on a rack and incubated in standard conditions. Half of the differentiation media was replaced every other day.

Myoblast-cell Line Culture and Differentiation:

Myoblasts (C2C12 from ATCC, CRL 1772) were cultured in in Myoblast medium (DMEM with 20% FBS) up to approximately 50% confluency. Cells were detached for passaging using TrypLE according to growth status. For differentiation, when cultures became fully confluent, were washed with PBS, and the above Myoblast medium was replaced by Myofiber differentiation media (DMEM, 0.5% FBS). Cell morphology was monitored using an IX51 inverted 100 microscope (Olympus).

Myoblast Primary Culture.

Primary myoblasts were isolated from 5-week old female mice. The hind limb 1 skeletal muscles were minced and digested in type-I collagenase and dispase B mixture. The digestion was stopped with F-10 Ham's medium containing 20% FBS, and the cells were filtered from debris, centrifuged and cultured in growth media (F-10 Ham's media, 17% FBS, 4ng/mL FGF2 and 1% penicillin-streptomycin) on uncoated dishes for three days when 5 mL growth media were added each 106 day. Then the supernatant was collected, centrifuged, and trypsinized with 0.25% trypsin. After washing off the trypsin, primary myoblasts were seeded on collagen-coated dishes, and the growth medium was changed every two days.

Mouse embryonic stem cell culture

ZHBTcH4 ESCs were cultured on gelatin-coated plates via standard medium containing fetal bovine serum and LIF. For trophectoderm differentiation, ZHBTcH4 ESCs were cultured in the presence of 2 μg/mL Dox to repress Oct-3/4 expression²⁷.

Human induced astrocytes (iAstrocytes)

First, to get neural precursor cells (NPCs), human iPSCs were dissociated into single cells by accutase, seeded at 20,000 cells/cm²density on matrigel-coated plates and cultured in mTESR1 medium containing 1:100 of Rock inhibitor overnight. Next day, the medium was switched to N2B27-medium (DMEM/F12, 1X N2, 1X B27, 1X Glutamax, 1X NEAA, β3116 mercaptoethanol [1:1000], and 25 μg/mL insulin), supplemented with the small molecules SB431542 10 μM and LDN193189 1μM. Medium was changed daily until day-8, at which time SB431542 and LDN193189 were withdrawn. On day-14, cells were dissociated and further maintained at high density, grown on matrigel in NPC-medium (DMEM/F12, 1X N2, 1X B27, and 20ng/m1FGF2) and split every week. Second, for NPC-Astrocyte differentiation, NPCs were plated at 15,000 cells/cm²density on matrigel-coated plates in NPC-medium containing 1:100 of Rock inhibitor. Following day after seeding, NPC-medium was switched to astrocyte medium (2% FBS and astrocyte growth supplement). Cells were fed every 2-days for 30-days. Cultures were passaged at a 90-95% confluency.

Human BJ-fibroblasts

BJ skin fibroblast cells were obtained from ATCC and grown in DMEM supplemented with 10% FBS, lx Glutamax, and 1X MEM-NEAA.

All cell cultures above described were maintained on standard incubation conditions: 37° C. in 5% CO2, 128 95% humidified air. Viability and cell number were determined as required by trypan blue and the TC10 129 Automated Cell Counter (BioRad).

Treatment of differentiated cells with Metabolite Induction Medium (MIM)

Cell cultures seeded on adherent conditions were kept on their respective differentiation media until they reach a mature phenotype (defined by the expression of markers specific to each cell type). When cultures reached around 80-90% confluence, the differentiation media was removed, cells were washed twice with PBS and the media was replaced with CI-MIM. The composition of C1-MIM includes up to 6 metabolites (details of the combinations used are specified in figure legends) including Methionine, Threonine, Glycine, Putrescine, Cysteine [5mM], and S-adenosylmethionine [0.05mM], which are dissolved in a base medium (BM) composed by DMEM-F12 plus Neurobasal [1:1], supplemented with 1X B27, 1X N2. Due to the absence of normal serum concentrations (e.g., 10-20%), either the growth factor FGF2 (20 ng/mL) or reduced concentrations of serum, were used according to cell type (FGF2 was added in cultures of chondrocytes, astrocytes, neurons; while it did not support the viability in myoblasts, where 2% of serum was used instead. Similarly, in human fibroblasts, the reduction of serum was enough without any added cytokine to keep viable cultures). In all cases, from the same batch of each cell culture, a control culture was also washed with PBS but maintained with their own differentiation media along all the time of the treatment. Cells in this schema were fed every other day. Cells were monitored daily to attest to the change in morphology. Cells were collected on the fifth day or earlier after starting the MIM treatment (indicated in each specific case in the figure legend) for further analyses, as indicated in the figure legends of each experimental approach. Viability of cultures at the time of collection was measured by trypan blue, and all cases above 85% viable cells were processed for downstream analyses.

Transdifferentiation experiments plus C1-MIM treatment

For transdifferentiate BJ-fibroblast into neurons, the first cell type was transduced with lentivirus containing doxycycline-inducible Neurogenin and rTA3. Two days post-transduction cells were plated in desired density and treated with metabolites cocktail (MIM4 or MIM6) for 5-days followed by the addition of doxycycline at a concentration of 0.5 μg/mL for induction of Neurogenins for 3-days, then collecting the cells for RNA analysis.

Methods were adapted from Busskamp et al.⁵⁶. For transdifferentiate MSCs to myocytes, we constructed a MyoD overexpression AAV vector by inserting MyoD-2A-GFP cDNA into an AAV vector (AAV2 inverted terminal repeat vector) under the control of CAG promoter. The recombinant AAV vector was pseudo-typed with AAV-DJ capsid, and the viral particles were generated following the procedures of the Gene Transfer Targeting and Therapeutics Core at the Salk Institute for Biological Studies. We initiated the transdifferentiation of MSCs to myocytes by adding 1×10\hat{ }9 GC AAV-MyoD-2A-GFP and metabolites to the differentiation medium (DMEM with 2% FBS). The control cells were only treated with 1×10\hat{ }9 GC AAV-MyoD-2A GFP. At day 4, 6, and 8 post-differentiation, the cells were fixed with 4% PFA and processed for immunofluorescence. Myocytes were recognized by the myosin heavy chain, which was labeled by MF20.

Immunocytochemistry

For NSCs, astrocytes, neurons, or cerebellum-derived cells, those were seeded on Poly-D-Lysine coverslips, washed with PBS, and fixed in 4% PFA (15min). Samples were permeabilized and blocked for 1 h in 5% BSA+0.02% TritonX100; afterward, the primary antibody solution was added in PBS, and samples were kept in a wet chamber overnight. The next day, samples were washed with PBS+0.2% Tween20 and incubated with a secondary antibody solution in PBS for lh. DAPI-Vectashield was used to mount the samples. For myoblasts, after fixation, cells were blocked with 5% goat serum, 2% 175 BSA, 0.2% Triton X-100, and 0.1% sodium azide in PBS for at least lh; then the samples were incubated with primary antibodies overnight. After washing with PBS, the samples were incubated with respective secondary antibodies and DAPI for 45min at room temperature. Images were acquired using a Zeiss LSM 710 Laser Scanning Confocal Microscope (Zeiss). For quantification purposes, the percentage of cells positive to each marker was calculated regarding the total cell number identified by DAPI nuclei, from at least five pictures obtained from each sample. Images were processed with NIH ImageJ software. Primary antibodies used include Anti-beta-III-tubulin from SIGMA (Cat. T2200), Anti-GFAP from Abcam (Cat. ab4674), anti-Nestin from Millipore (MAB353), anti-Pax7 and anti-MF20 from DSHB (Cat. AB_528428 and AB_2147781), anti-MyoD from Santa Cruz (Cat. sc-377460), anti-Ki67 from Cell Signaling (Cat. 12202), Alexa-Fluor 568 and 488 (Cat. A10042, A21134, A11039, and A21206).

RNA isolation and gene expression analysis by rt-PCR

Total RNA was isolated from cells grown on Petri dish at the indicated time points, using the RNeasy Plus Mini kit QIAGEN, according to the manufacturer's protocol, including a DNA-removal step with DNAseI. Amount and purity of RNA were assessed using a NanoDrop spectrophotometer (Nanodrop Technologies); at least 500 ng of total RNA was used to synthesize cDNA by reverse transcription, using Maxima™ H

Minus cDNA Synthesis Master Mix. 2.5-10 ng of cDNA was used in the following qPCR performed on a CFX384 thermal cycler (Bio-Rad) using the SsoAdvanced™ Universal SYBR® Green Supermix. Results were normalized to at 193 least one reference genes β-Actin, RPL38, GAPDH, Gus, CTCF, and Natl, specified per figure), selected for their highest stability among a pool of common housekeeping genes. Primers were designed by NCBI/Primer-BLAST primers designing tool (Table 1). Statistical analysis of the results was performed using the 2ACt method^{s}. Results were expressed relative to the expression values of the experimental control.

TABLE 1
List of primers used in the study
(sequence for mouse, except
where indicated ‘human’)

| Gene | | |
|---|---|---|
| name | Forward primer seq | Revers primer seq |
| β-Actin | CATTGCTGACAG | TGCTGGAAGGTG |
| | GATGCAGAAGG | GACAGTGAGG |
| | (SEQ ID | (SEQ ID |
| | NO: 1) | NO: 2) |
| GAPDH | CATCACTGCCAC | ATGCCAGTGAGC |
| | CCAGAAGACTG | TTCCCGTTCAG |
| | (SEQ ID | (SEQ ID |
| | NO: 3) | NO: 4) |
| Nat1 | ATTCTTCGTTGT | AGTTGTTTGCTG |
| | CAAGCCGCCAAA | CGGAGTTGTCAT |
| | GTGGAG | CTCGTC |
| | (SEQ ID | (SEQ ID |
| | NO: 5) | NO: 6) |
| RPL38 | AGGATGCCAAGT | TCCTTGTCTGTG |
| | CTGTCAAGA | ATAACCAGG |
| | (SEQ ID | (SEQ ID |
| | NO: 7) | NO: 8) |
| Ascl1 | CTCGTCCTACTC | ATCTGCTGCCAT |
| | CTCCGACG | CCTGCTTC |
| | (SEQ ID | (SEQ ID |
| | NO: 9) | NO: 10) |
| Cd44 | ACAACCCTTCAG | CGCCGCTCTTAG |
| | CCTACTGC | TGCTAGAT |
| | (SEQ ID | (SEQ ID |
| | NO: 11) | NO: 12) |
| Cd9 | CTGTGGCATAGC | AGACCTCACTGA |
| | TGGTCCTTTG | TGGCTTCAGG |
| | (SEQ ID | (SEQ ID |
| | NO: 13) | NO: 14) |
| cMyc | GTGCTGCATGAG | GACCTCTTGGCA |
| | GAGACACC | GGGGTTTG |
| | (SEQ ID | (SEQ ID |
| | NO: 1) | NO: 16) |
| Klf4 | GCACACCTGCGA | CCGTCCCAGTCA |
| | ACTCACAC | CAGTGGTAA |
| | (SEQ ID | (SEQ ID |
| | NO: 17) | NO: 18) |
| GFAP | AGGTTGAATCGC | GCTTGGCCACAT |
| | TGGAGGAG | CCATCTC |
| | (SEQ ID | (SEQ ID |
| | NO: 19) | NO: 20) |
| Nes | CCAGAGCTGGAC | ACCTGCCTCTTT |
| | TGGAACTC | TGGTTCCT |
| | (SEQ ID | (SEQ ID |
| | NO: 21) | NO: 22) |
| CD133 | TCCCTCCTGTGC | CCAAACTTCTTC |
| | AGCAATCA | GTTTCCCCGA |
| | (SEQ ID | (SEQ ID |
| | NO: 23) | NO: 24) |
| Sox2 | AACGGCAGCTAC | CGAGCTGGTCAT |
| | AGCATGATGC | GGAGTTGTAC |
| | (SEQ ID | (SEQ ID |
| | NO: 25) | NO: 26) |
| Hes5 | CCGTCAGCTACC | GGTCAGGAACTG |
| | TGAAACACAG | TACCGCCTC |
| | (SEQ ID | (SEQ ID |
| | NO: 27) | NO: 28) |
| Mtr | GCTCTGTGAAGA | GAGCCATTCCTC |
| | CCTCATCTGG | CACTCATCTG |
| | (SEQ ID | (SEQ ID |
| | NO: 29) | NO: 30) |
| Chrd1 | GTATGCAGAGGG | TGGAGGATCGTA |
| | GATGCAGAA | GGGGGAAC |
| | (SEQ ID | (SEQ ID |
| | NO: 31) | NO: 32) |
| Ahcy | CAGGCTATGGTG | CCTCCTTACAGG |
| | ATGTGGGCAA | CTTCGTCCAT |
| | (SEQ ID | (SEQ ID |
| | NO: 33) | NO: 34) |
| Odc1 | TGCCACACTCAA | ACACTGCCTGAA |
| | AACCAGCAGG | CGAAGGTCTC |
| | (SEQ ID | (SEQ ID |
| | NO: 35) | NO: 36) |
| Beta-III- | ACCTATTCAGGC | GCAGGCAGTCAC |
| tubulin | CCGACAACTTTA | AATTCTCACAC |
| | (SEQ ID | (SEQ ID |
| | NO: 37) | NO: 38) |
| Map2 | CTGCGAGTAAGC | AGCTGAGGAACC |
| | TGTGACCG | TTAATTCTTGCC |
| | (SEQ ID | (SEQ ID |
| | NO: 39) | NO: 40) |
| SB100 | GACTCCAGCAGC | TTGATTTCCTCC |
| | AAAGGTGA | AGGAAGTGAGAG |
| | (SEQ ID | (SEQ ID |
| | NO: 41) | NO: 42) |
| Mat1a | CCTTCTCTGGAA | GACAGAGGTTCT |
| | AGGACTACACC | GCCACACCAA |
| | (SEQ ID | (SEQ ID |
| | NO: 43) | NO: 44) |
| Ascl1 | CGGAACTGATGC | GGCAAAACCCAG |
| | GCTGCAAACG | GTTGACCAAC |
| | (SEQ ID | (SEQ ID |
| | NO: 45) | NO: 46) |
| Sox5 | CGCCAGATGAAA | TGAGTCAGGCTC |
| | GAGCAACTCAG | TCCAGTGTTG |
| | (SEQ ID | (SEQ ID |
| | NO: 47) | NO: 48) |
| Cd51 | GTGTGAGGAACT | CCGTTCTCTGGT |
| | GGTCGCCTAT | CCAACCGATA |
| | (SEQ ID | (SEQ ID |
| | NO: 49) | NO: 50) |
| Thy1 | CCTTACCCTAGC | TTATGCCGCCAC |
| | CAACTTCACC | ACTTGACCAG |
| | (SEQ ID | (SEQ ID |
| | NO: 51) | NO: 52) |
| Sox9 | GCAGACCAGTAC | CTCGTTCAGCAG |
| | CCGCATCT | CCTCCAG |
| | (SEQ ID | (SEQ ID |
| | NO: 53) | NO: 54) |
| Aggrecan | CCTGCTACTTCA | AGATGCTGTTGA |
| | TCGACCCC | CTCGAACCT |
| | (SEQ ID | (SEQ ID |
| | NO: 55) | NO: 56) |
| Col2a | AATGGGCAGAGG | CATTCCCAGTGT |
| | TATAAAGATAAG | CACACACACA |
| | GA | (SEQ ID |
| | (SEQ ID | NO: 58) |
| | NO: 57) | |
| Oct4 | CAGCAGATCACT | |
| | CACATCGCCA | GCCTCATACTCT |
| | (SEQ ID | TCTCGTTGGG |
| | NO: 59) | (SEQ ID |
| Myf5 | TATTACAGCCTG | CTGCTGTTCTTT |
| | CCGGGACA | CGGGACCA |
| | (SEQ ID | (SEQ ID |
| | NO: 61) | NO: 62) |
| Ccnd1 | AGCCTCCAGAGG | TGGGGAGGGCTG |
| | GCTGTCGG | TGGTCTCG |
| | (SEQ ID | (SEQ ID |
| | NO: 63) | NO: 64) |
| Myhllb | TCAATGAGATGG | GTCCAGGTGCAG |
| | AGATCCAGCTGA | CTGTGTGTCCTT |
| | AC | C |
| | (SEQ ID | (SEQ ID |
| | NO: 65) | NO: 66) |
| MyoG | GCAGGCTCAAGA | TAGGCGCTCAAT |
| | AAGTGAATGA | GTACTGGAT |
| | (SEQ ID | (SEQ ID |
| | NO: 67) | NO: 68) |
| Cmk | AGACAAGCATAA | AGGCAGAGTGTA |
| | GACCGACCT | ACCCTTGAT |
| | (SEQ ID | (SEQ ID |
| | NO: 69) | NO: 70) |
| Mef2c | ATCCCGATGCAG | AACAGCACACAA |
| | ACGATTCAG | TCTTTGCCT |
| | (SEQ ID | (SEQ ID |
| | NO: 71) | NO: 72) |
| Ef1af | AGCTTCTCTGAC | GACCGTTCTTCC |
| | TACCCTCCACTT | ACCACTGATT |
| | (SEQ ID | (SEQ ID |
| | NO: 73) | NO: 74) |
| Human | GGTTTCACCAGG | ACTCTCGTCGGT |
| GUS B | ATCCACCTC | GACTGTTC |
| | (SEQ ID | (SEQ ID |
| | NO: 75) | NO: 76) |
| Human | AATCCCATCACC | TGGACTCCACGA |
| GAPDH | ATCTTCCA | CGTACTCA |
| | (SEQ ID | (SEQ ID |
| | NO: 77) | NO: 78) |
| Human | CAAAAATGGCCA | AGTTGGGATCGA |
| SOX2 | TGCAGGTT | ACAAAAGCTATT |
| | (SEQ ID | (SEQ ID |
| | NO: 79) | NO: 80) |
| Human | AGAGATCCGCAC | GTAGTCGTTGGC |
| GFAP | GCAGTATG | TTCGTGCT |
| | (SEQ ID | (SEQ ID |
| | NO: 81) | NO: 82) |
| Human | GGATTCTCTGCT | AGACTCTGACCT |
| cMYC | CTCCTCGAC | TTTGCCAGG |
| | (SEQ ID | (SEQ ID |
| | NO: 83) | NO: 84) |
| Human | CACTACCAAGGA | CAACGCCTCTTT |
| CD133 | CAAGGCGTTC | GGTCTCCTTG |
| | (SEQ ID | (SEQ ID |
| | NO: 85) | NO: 86) |
| Human | CCTGAAGCAGAA | AAAGCGGCAGAT |
| OCT4 | GAGGATCACC | GGTCGTTTGG |
| | (SEQ ID | (SEQ ID |
| | NO: 87) | NO: 88) |
| Human | CATCTCAAGGCA | TCGGTCGCATTT |
| KLF4 | CACCTGCGAA | TTGGCACTGG |
| | (SEQ ID | (SEQ ID |
| | NO: 89) | NO: 90) |
| Human | AAATACCTCAGC | CCATTGCTATTC |
| NANOG | CTCCAGCAG | TTCGGCCAG |
| | (SEQ ID | (SEQ ID |
| | NO: 91) | NO: 92) |
| Human | GGCCCTCAAGGT | CACCCTGTGGTC |
| COL1A2 | TTCCAAGG | CAACAACTC |
| | (SEQ ID | (SEQ ID |
| | NO: 94) | NO: 95) |

Knockdown Experiments

Small interference RNA (siRNA) for Mtr and Matla were prepared according to the provider's instructions. Briefly, oligos were adjusted to 100 μM concentration in RNAse free water. Then, cells plated over adherent conditions (on 6-well plates) were transfected using the magnetofection method (Lipofectamine with Combimag) and scaling the volume at 1 mL/well, for 24 hours. Then collection or differentiation was performed according to the experimental needs, as figures indicate.

Relative measurement of methionine and S-adenosylmethionine Cells treated under the respective conditions (as indicated in the figures) were collected (at least 250,000 207 cells per each technical replicate). Pellets were flash-frozen and stored in LN2-tank until processed. We lysed and processed the cell pellets according to kit's manufacturer instructions either for methionine quantification (Methionine Assay Kit, fluorometric, Em/Ex: 535/587) or for SAM quantification (S210 Adenosylmethionine ELISA Kit, colorimetric, 450nm).

Metabolome Analysis

Sample Collections:

Cells and media were collected according to the required time as NSCs, MSCs, and Myoblasts or during their respective differentiation conditions, in order to obtain fingerprint (intracellular) and footprint (extracellular) readings. Each sample was derived from a cell pellet of 100 pL mass-volume measured by the Eppendorf microtube scale. After the indicated time, cell metabolism was stopped by placing cells on an ice-bed, where cell collection was performed. Cells were scraped from wells, centrifuged 300g×5min at 4° C., and pellets were flash-frozen in LN2 until further processing by Metabolon® company.

Processing of samples at Metabolon®: Briefly, samples were homogenized and subjected to methanol extraction then split into aliquots for analysis by ultrahigh performance liquid chromatography/mass spectrometry (UHPLC/MS) in the positive (two methods) and negative (two methods) mode. Metabolites were then identified by an automated comparison of ion features to a reference library chemical standards followed by visual inspection for quality control⁵⁸. For statistical analyses and data display, any missing values are assumed to be below the limits of detection. Statistical tests were performed in ArrayStudio (Omicsoft) or “R” to compare data between experimental groups; P<0.05 is considered significant and 0.05<P<0.10 to be trending. An estimate of the false discovery rate (Q-value) was also calculated to take into account the multiple comparisons that normally occur in metabolomic-based studies, with q<0.05 used as an indication of high confidence as a result.

Determination of Metabolomic Patterns:

The relative intracellular abundance of metabolites was estimated as Scaled Intensity, where each value is normalized by Bradford protein concentration before being considered (n=5 biological replicates). Then, the calculated averages of the scaled intensity of 5 biological replicates per condition were plotted (on axes ‘y’) against a continuous scale of time (on axes ‘x’) according to the collection times determined for each cell type (data not shown). The plots were classified and sorted according to fixed parameters by the Recurrent Pattern Classification Strategy.

Enrichment Analyses: The analyses were performed using the Enrichment Analysis tool of 238 MetaboAnalyst® (www.metaboanalyst.ca), where we used only the metabolites recognized by the 239 Human Metabolome Data Base (HMDB IDs), with the library Pathway-associated metabolite sets 240 (SMPDB)⁵⁹).

Heatmaps, hierarchical cluster analysis (HCA), K-means clustered analysis and Silhouette analysis:

Data used correspond to the values of Bradford-normalized median-Scaled value of each metabolite from 5 biologically replicates. Those analyses were conducted using the MeV software, as previously described⁶⁰. For the clustering analysis of the metabolites profile, the k-means algorithm was performed in R. Briefly, normalized metabolite values for each cell samples were analyzed using the silhouette method to determine the optimal number of clusters. Then, the K-means algorithm was performed with the optimal number of clusters to group the metabolites based on the patterns in metabolite expression levels.

Bump chart analysis. Mean values of the Bradford-normalized median-Scaled of each metabolite were subjected to analyze the prevalence of metabolic pathways at each time point. Top-20 enriched metabolic pathways were analyzed based on the summarized value according to sub-pathways from super pathways as provided by the Metabolon® database. The representations were obtained using Excel, GraphPad Prism 8 software, RAW graphs software, and Adobe Illustrator..

Bulk RNA-sequencing

Total-RNA was derived from cell cultures and extracted using 1 mL TRIZOL following the manufacturer's protocol. The RNA concentration was measured using a synergy Hl-Biotek. RNA integrity was determined using a TapeStation RNA system (Agilent). cDNA was prepared using Illumina TruSeq kit (Cat. 20020594). The samples were run in biological triplicates as single end 100 bp on a HiSeq4000 (Illumina). Illumina reads were processed by FastQC quality control (www.bioinformatics.babraham.ac.uk/projects/fastqc/), trimming adapter sequences with Cutadapt tools⁶¹ then remaining reads were mapped to the 62 respective reference mouse genome(mm10) using HISAT2 2.1.0⁶². We quantified gene expression from the mapped reads using HTSeq-count (www huber.embl.de/users/anders/HTSeq/doc/count.html) that obtained integer counts of mapped reads per gene. Cufflinks v2.2.1 was used to obtain FPKM expression values, using an automatic estimation of the library size distributions and sequence composition bias correction⁶³. Differentially expressed genes were identified based on integer count data using R package

DESeq2 version 1.22.2⁶⁴, which determines 268 DE by modeling count data using a negative binomial distribution as follows: First, size factors are calculated to take into account the total number of reads in different samples. Second, a dispersion parameter is determined for each gene, which accounts for biological variation between samples. Third, a negative binomial distribution is used to fit the counts for each gene. The P-value is calculated based on the wald test. The P values adjusted for multiple testing were calculated using the BenjaminiHochberg procedure, which controls the false discovery rate (FDR<0.05). Volcano plots are based on the bcbioRNAseq R package⁶⁵. For the list of differentially mRNA genes, we tested whether each had enriched GO terms in biological process and molecular functions using the ToppGene Suite ⁶⁶ and Gene Set Enrichment Analysis (GSEA) software^{36,37}. For ToppGene, only those functional annotation terms associated with the various sets of differentially expressed genes were clustered that were significantly enriched (Bonferroni correction, p<0.01) compared with the function annotation terms associated with the total population of genes. For GSEA, we consider function enrichment based on NES having an FDR-q value below 0.25.

scRNA-Sequencing

Single-cell suspensions were collected by harvesting cell cultures by trypsinization. Cells were washed into 4° C. PBS and pelleted by spinning at 300g, 5 min, at 4° C. This wash step was repeated two more times. After final washing, 200μL of cold-PBS were added to 1×10\hat{ }6 cells. For cell fixation , 800μL of methanol were added dropwise, and the samples were incubated for 30 min at −20° C., then stored at 80° C. until further processing. Fixed cells were equilibrated on ice for 5min and pelleted at 1,000g for 5min 4° C. The supernatant was carefully removed, and cells were resuspended into 0.04% BSA, 1mM 289 DTT, 0.2U/mL RNase Inhibitor in 3× SSC buffer at approx. 1000 cells/mL. Cells were processed for 290 single-cell sequencing on a 10× genomics system according to the manufacturer's protocol. CellRanger v3.0.2 software was used to align reads to the 10× Genomics pre-built mm10 reference genome for astrocytes, MIM-astrocytes, and NSCs (Neural Stem Cells) datasets with the default setting for de-multiplexing to generate feature-barcode matrix. The R package Seurat v3.1.1⁶⁸ was used to read and analyze feature-barcode matrix following the steps: First, we filtered the cells that have unique feature counts according to quality control matrix plots, and after filtering, we have astrocytes 9055 cells, MIM-astrocytes 14911 cells, and NSCs 8985 cells; then, UMI counts were normalized with NormalizeData function using default settings. Seurat's RunUMAP function was used to do non-linear dimension reduction and cluster with resolution setting as 0.2. Differentially expressed genes or conserved markers in the clusters between astrocytes and MIM-astrocytes or between MIM-astrocytes and NSCs were aligned by the Seurat integrative analysis. In detail, the FindlntegrationAnchors function was first used to identify anchors which are representing cells sharing similar biological states based on canonical correlation analysis; then, analysis integrated (of all cells) was performed following the Seurat package, step by step⁴¹. For the differentially expressed genes, studies tested whether each had enriched GO terms in biological process and molecular functions using the ToppGene Suite⁶⁶. For Pearson correlation analysis between single-cell and bulk-RNAseq, the gene expression fold change values (logFC) were considered, from matching comparisons. For example, the differential expressed genes from the comparison NSC vs. MIM-astrocytes, require: (a) the differential expressed genes fold change values logFC, from the cluster comparison in scRNAseq (like NM4, which compares NSCs and MIM-astrocytes in single-cell data), and (b) the differential expressed genes fold change values logFC, from the genes identified in (a) (i.e., between NSCs and MIM-astrocytes) but derived from bulk-RNAseq. For cell identification analysis, single-cell transcriptomes were extracted from the MIM astrocyte's dataset (which has 14911 cells). Then, MIM-astrocytes single-cell transcriptomes were applied to the interested cell atlas as listed in http://scibet.cancer-pku.cni, to predict or classify the cell types in each cluster following the steps provided by Scibet⁶⁹. Lastly, the cell types obtained were summarized with a probability above 0.8 for cell type prediction accuracy. A second classification system was computed, following an assessment method previously published for OSKM-treated astrocytes⁴². In addition, gene expression levels of the published conventional gene markers for the brain cell were analyzed to assess the cell type of the metabolite-treated astrocytes. For trajectory analysis, single-cell transcriptomes were extracted from the MIM-astrocyte's dataset and analyzed by using the package Monocle^{70,71} as indicated.

Methylation Analysis with Reduced Representation Bisulfite Sequencing

NSCs and NSCs undergoing differentiation were collected at the times indicated in the respective figure. Briefly, cells were detached and washed with 10 mL chilled PBS, then recovered by centrifugation at 800g, 4° C. Cell pellets (above 5×10\hat{ }5 cells) were frozen directly on dry ice and stored at −80° C. Downstream processes including gDNA isolation, quantification, digestion, adaptor ligation, bisulfite 329 conversion, library generation, next-generation sequencing (using Illumina platform), and data analysis 330 were carried out by Active Motif, Inc.

Quantification of Histone Modifications

Bulk histones were acid-extracted from cell pellets, propionylated, and subjected to trypsin digestion. Histone peptides were resuspended in 0.1% TFA in H2O for mass spectrometry analysis. Samples were analyzed on a triple quadrupole (QqQ) mass spectrometer (Thermo Fisher Scientific TSQ Quantiva) directly coupled with an UltiMate 3000 Dionex nano-liquid chromatography system. Targeted analysis of unmodified and various modified histone peptides was performed. This entire process was repeated three separate times for each sample. Data were imported and analyzed in Skyline with Savitzky-Golay smoothing. Each modification was represented as a percentage of the total pool of modifications. The 340 process from the histone extraction to the analysis was carried out by Active Motif, Inc.

Chromatin Immunoprecipitation (ChIP)-qPCR

ChIP reactions were performed using 30pg of chromatin and 4pg of H3K27me3 antibody (Active Motif, 344 Cat. 39155). Subsequent qPCR was ran using one positive control primer pair for the histone mark that worked well in similar assays (Gapdh, Hoxcl0), the regions of interest, as well as a negative control 346 primer pair that amplifies for the promoter region of the active gene Actb. PCR-reactions were set up in triplicate for each ChIP sample. Each qPCR plate also contained input DNA and a standard curve for normalization. Normalized data is expressed as Binding Events Detected per 1000 Cells. The entire process from chromatin extraction to the analysis was carried out by Active Motif, Inc.

Statistical analyses

All data is shown as means ±S.D. or S.E.M, as indicated in each figure legend. Statistics were performed using GraphPad Prism Software. Comparisons between two groups were analyzed using t-Test Twotailed, or one-way ANOVA followed by either Bonferroni or Turkey's post hoc test, as appropriate. The statistics from metabolome analyses were obtained under the Metabolon Portal Software (www.portal.metabolon.com/en). For transcriptomic analyses, R Software (R version 3.5.1) was used for statistics, other details about these analyses in their respective methods' section. For all experiments, values of P<0.05 were considered statistically significant. No statistical methods were used to predetermine the sample size. The experiments were not randomized. The investigators were blinded in some but not all experiments.
Supplementary Information SI_2

Extended Methods of Metabolon®

The datasets comprise compounds of known identity. Following normalization to Bradford protein concentration, log transformation and imputation of missing values, if any, with the minimum observed value for each compound, ANOVA contrasts and Welch's two-sample t-test was used to identify biochemicals that differed significantly between experimental groups. Analysis by one-way ANOVA identified biochemicals exhibiting significant group effect.

An estimate of the false discovery rate (q-value) is calculated to take into account the multiple comparisons that normally occur in metabolomic-based studies. For example, when analyzing 200 compounds, we would expect to see about 10 compounds meeting the p≤0.05 cut-off by random chance. The q-value describes the false discovery rate; a low q-value (q<0.10) is an indication of high confidence in a result. While a higher q-value indicates diminished confidence, it does not necessarily rule out the significance of a result. Other lines of evidence may be taken into consideration when determining whether a result merits further scrutiny. Such evidence may include a) significance in another dimension of the study, b) inclusion in a common pathway with a highly significant compound, or c) residing in a similar functional biochemical family with other significant compounds. Refer to the Appendix for further descriptions of procedures performed at Metabolon®.

Appendix of Metabolon Platform

Sample Accessioning: Following receipt, samples were inventoried and immediately stored at −80° C. Each sample received was accessioned into the Metabolon LIMS system and was assigned by the LIMS a unique identifier that was associated with the original source identifier only. This identifier was used to track all sample handling, tasks, results, etc. The samples (and all derived aliquots) were tracked by the LIMS system. All portions of any sample were automatically assigned their own unique identifiers by the LIMS when a new task was created; the relationship of these samples was also tracked. All samples were maintained at −80° C. until processed. Sample Preparation: Samples were prepared using the automated

MicroLab STAR® system from Hamilton Company. Several recovery standards were added prior to the first step in the extraction process for QC purposes. To remove protein, dissociate small molecules bound to protein or trapped in the precipitated protein matrix, and to recover chemically diverse metabolites, proteins were precipitated with methanol under vigorous shaking for 2 min (Glen Mills GenoGrinder 2000) followed by centrifugation. The resulting extract was divided into five fractions: two for analysis by two separate reverse phase (RP)/UPLC-MS/MS methods with positive ion mode electrospray ionization (ESI), one for analysis by RP/UPLC-MS/MS with negative ion mode ESI, one for analysis by HILIC/UPLC-MS/MS with negative ion mode ESI, and one sample was reserved for backup. Samples were placed briefly on a TurboVap® (Zymark) to remove the organic solvent. The sample extracts were stored overnight under nitrogen before preparation for analysis.

QA/QC: Several types of controls were analyzed in concert with the experimental samples: a pooled matrix sample generated by taking a small volume of each experimental sample (or alternatively, use of a pool of well-characterized human plasma) served as a technical replicate throughout the data set; extracted water samples served as process blanks; and a cocktail of QC standards that were carefully chosen not to interfere with the measurement of endogenous compounds were spiked into every analyzed sample, allowed instrument performance monitoring and aided chromatographic alignment. Tables 2 and 3 (below) describe these QC samples and standards. Instrument variability was determined by calculating the median relative standard deviation (RSD) for the standards that were added to each sample prior to injection into the mass spectrometers. Overall process variability was determined by calculating the median RSD for all endogenous metabolites (i.e., non-instrument standards) present in 100% of the pooled matrix samples. Experimental samples were randomized across the platform run with QC samples spaced evenly among the injections.

TABLE 2
Description of Metabolon QC Samples

| Type | Description | Purpose |
|---|---|---|
| MTRX | Large pool of human | Assure that all aspects of the |
| | plasma maintained by | Metabolon process are operating |
| | Metabolon that has been | within specifications. |
| | characterized extensively. | |
| CMTRX | Pool created by taking a | Assess the effect of a non- |
| | small aliquot from every | plasma matrix on the Metabolon |
| | customer sample. | process and distinguish |
| | | biological variability from |
| | | process variability. |
| PRCS | Aliquot of ultra-pure water | Process Blank used to assess |
| | | the contribution to compound |
| | | signals from the process. |
| SOLV | Aliquot of solvents used in | Solvent Blank used to segregate |
| | extraction. | contamination sources in the |
| | | extraction. |

TABLE 3
Metabolon QC Standards

| Type | Description | Purpose |
|---|---|---|
| RS | Recovery Standard | Assess variability and verify performance |
| | | of extraction and instrumentation. |
| IS | Internal Standard | Assess variability and performance of |
| | | instrument. |

Ultrahigh Performance Liquid Chromatography-Tandem Mass Spectroscopy (UPLC-MS/MS): All methods utilized a Waters ACQUITY ultra-performance liquid chromatography (UPLC) and a Thermo Scientific Q-Exactive high resolution/accurate mass spectrometer interfaced with a heated electrospray ionization (HESI-II) source and Orbitrap mass analyzer operated at 35,000 mass resolution. The sample extract was dried then reconstituted in solvents compatible to each of the four methods. Each reconstitution solvent contained a series of standards at fixed concentrations to ensure injection and chromatographic consistency. One aliquot was analyzed using acidic positive ion conditions, chromatographically optimized for more hydrophilic compounds. In this method, the extract was gradient eluted from a C18 column (Waters UPLC BEH C18-2.1×100 mm, 1.7 μm) using water and methanol, containing 0.05% perfluoropentanoic acid (PFPA) and 0.1% formic acid (FA). Another aliquot was also analyzed using acidic positive ion conditions; however, it was chromatographically optimized for more hydrophobic compounds. In this method, the extract was gradient eluted from the same afore mentioned C18 column using methanol, acetonitrile, water, 0.05% PFPA and 0.01% FA and was operated at an overall higher organic content. Another aliquot was analyzed using basic negative ion optimized conditions using a separate dedicated C18 column. The basic extracts were gradient eluted from the column using methanol and water, however with 6.5mM Ammonium Bicarbonate at pH 8. The fourth aliquot was analyzed via negative ionization following elution from a HILIC column (Waters UPLC BEH Amide 2.1×150 mm, 1.7 μm) using a gradient consisting of water and acetonitrile with 10mM Ammonium Formate, pH 10.8. The MS analysis alternated between MS and data-dependent MSn scans using dynamic exclusion. The scan range varied slighted between methods but covered 70-1000 m/z. Raw data files are archived and extracted as described below.

Bioinformatics: The informatics system consisted of four major components, the Laboratory Information Management System (LIMS), the data extraction and peak-identification software, data processing tools for QC and compound identification, and a collection of information interpretation and visualization tools for use by data analysts. The hardware and software foundations for these informatics components were the LAN backbone, and a database server running Oracle 10.2.0.1 Enterprise Edition.

LIMS: The purpose of the Metabolon LIMS system was to enable fully auditable laboratory automation through a secure, easy to use, and highly specialized system. The scope of the Metabolon LIMS system encompasses sample accessioning, sample preparation and instrumental analysis and reporting and advanced data analysis. All of the subsequent software systems are grounded in the LIMS data structures. It has been modified to leverage and interface with the in-house information extraction and data visualization systems, as well as third party instrumentation and data analysis software.

Data Extraction and Compound Identification: Raw data was extracted, peak-identified and QC processed using Metabolon's hardware and software. These systems are built on a web-service platform utilizing Microsoft's .NET technologies, which run on high-performance application servers and fiber-channel storage arrays in clusters to provide active failover and load-balancing. Compounds were identified by comparison to library entries of purified standards or recurrent unknown entities. Metabolon maintains a library based on authenticated standards that contains the retention time/index (RI), mass to charge ratio (m/z), and chromatographic data (including MS/MS spectral data) on all molecules present in the library. Furthermore, biochemical identifications are based on three criteria: retention index within a narrow RI window of the proposed identification, accurate mass match to the library +/−10 ppm, and the MS/MS forward and reverse scores between the experimental data and authentic standards. The MS/MS scores are based on a comparison of the ions present in the experimental spectrum to the ions present in the library spectrum. While there may be similarities between these molecules based on one of these factors, the use of all three data points can be utilized to distinguish and differentiate biochemicals. More than 3300 commercially available purified standard compounds have been acquired and registered into LIMS for analysis on all platforms for determination of their analytical characteristics. Additional mass spectral entries have been created for structurally unnamed biochemicals, which have been identified by virtue of their recurrent nature (both chromatographic and mass spectral). These compounds have the potential to be identified by future acquisition of a matching purified standard or by classical structural analysis.

Curation: A variety of curation procedures were carried out to ensure that a high-quality data set was made available for statistical analysis and data interpretation. The QC and curation processes were designed to ensure accurate and consistent identification of true chemical entities, and to remove those representing system artifacts, mis-assignments, and background noise. Metabolon data analysts use proprietary visualization and interpretation software to confirm the consistency of peak identification among the various samples. Library matches for each compound were checked for each sample and corrected if necessary.

Metabolite Quantification and Data Normalization: Peaks were quantified using area-under-the-curve. For studies spanning multiple days, a data normalization step was performed to correct variation resulting from instrument inter-day tuning differences. Essentially, each compound was corrected in run-day blocks by registering the medians to equal one (1.00) and normalizing each data point proportionately (termed the “block correction” For studies that did not require more than one day of analysis, no normalization is necessary, other than for purposes of data visualization. In certain instances, biochemical data may have been normalized to an additional factor (e.g., cell counts, total protein as determined by Bradford assay, osmolality, etc.) to account for differences in metabolite levels due to differences in the amount of material present in each sample.

Supplementary Information SI_4

Manifest about the customization of concentrations for C1-MIM cocktail

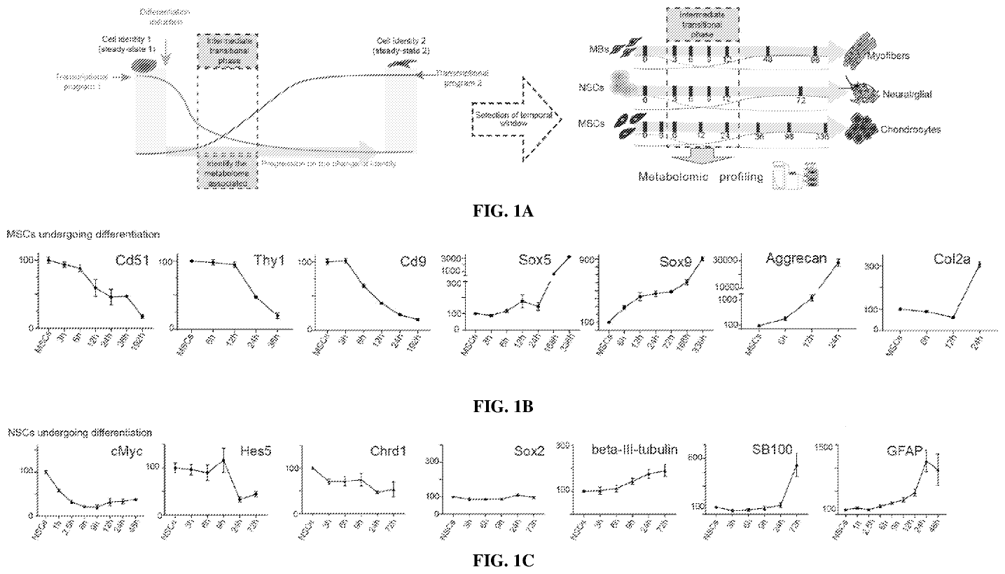

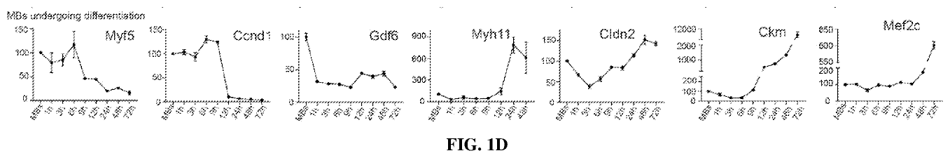

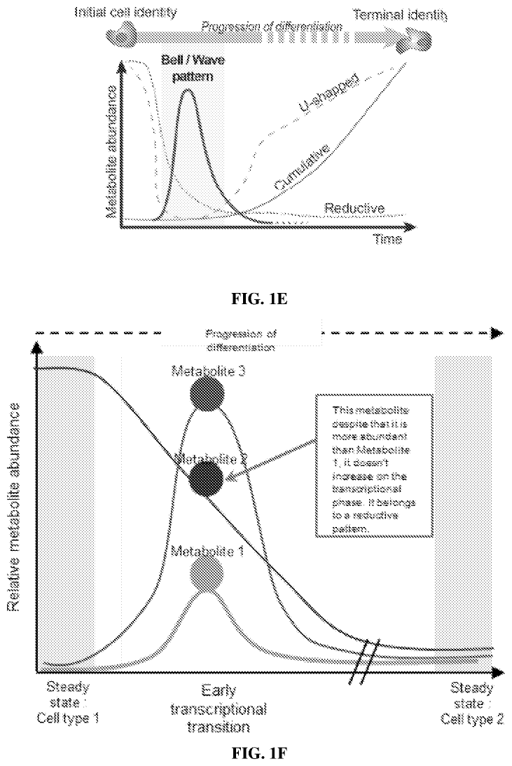

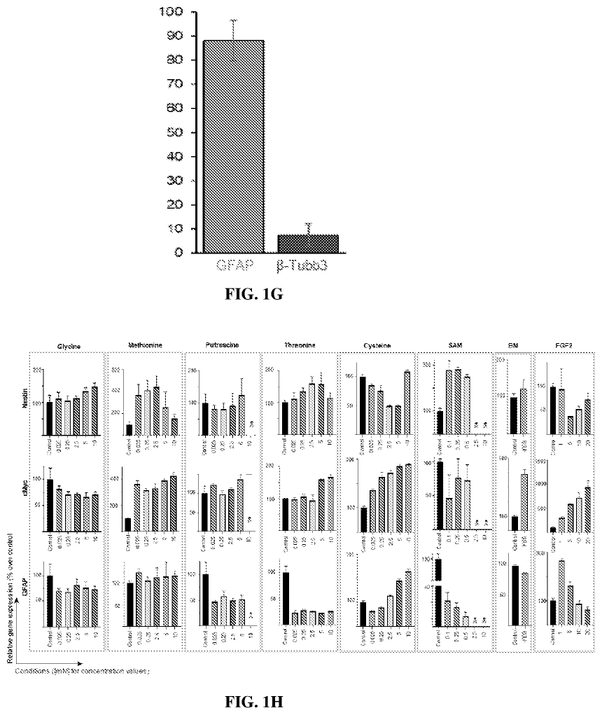

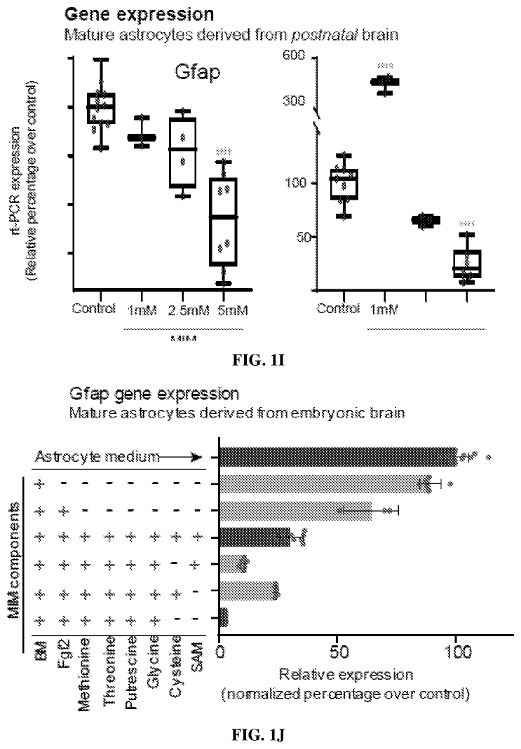

The cocktail customization was performed in primary cultures of mature astrocytes, between the second and the fourth passage. Those cells were maintained at least 8 days in culture before passaging. We corroborated the expression of Gfap before starting the experiment by immunocytochemistry (FIG. 1G). We tested individual metabolites at different concentrations in a serum-free medium (base media, BM). BM contained equivalent volumes of DMEM-F12 and Neurobasal, plus the supplements N2 and B27 at 1X. Notes about specific molecules added. S-adenosylmethionine (SAM) at millimolar concentrations was lethal for astrocytes, which is in line with the lower physiological concentrations usually found for this metabolite compared to others. Cysteine is high-sensitive to oxidation, which is manifested as white flake precipitation, a high-concentrated stock solution [2500mM] dissolved in ddH2O with pH slightly acid (5.5-6) was prepared to prevent this issue. We corroborated a pH always close to 7.4 in the cells maintained in vitro, before and after treating them with cocktails containing the diluted solution of cysteine.

Only for customization purposes, we observed the relative gene expression of Gfap, cMyc, and Nestin against the exposure of a range of concentrations of each of the components for C1-MIM (FIG. 1H). Gfap expression was the reference trait, as it is the primary marker of astrocytes. Nestin and cMyc are more related to the precursor stage or Neural Stem/Progenitor Cells. The tracking of this gene expression was performed only with the goal of observing trends to select an appropriate concentration, i.e., not lethal and with a discernible effect in gene expression compared with controls.

Because the growth factor FGF2 was added to improve survival, we also perform a curve of different concentrations of FGF2; of note, the selected concentration of 20ng/mL did not inhibit Gfap, and lower concentrations even potentiate the expression of Gfap (this effect is different from the overall effect of the C1-MIM cocktail repressing Gfap-expression). Because metabolites alone exhibited a similar effect in concentrations ranging from low to high, we then tested the combinatory effect. As we speculated that a potential optimal outcome could derive from the repression of the mature markers, we observed Gfap and Cd44, as reference. We tested the mixture of all elements at 1mM, 2.5mM, and 5mM, with the exception of SAM, which was provided at 0.1mM, 0.25mM, and 0.5mM, respectively (i.e., in a ten times less concentrated compared with the other metabolites). This cocktail represents the C1-MIM_6 with 6 metabolites (FIG. 1I). As observed, despite that the range of 1mM with separate metabolites like putrescine or threonine, repressed Gfap, when those were added in combination, the synergic result did not inhibit Gfap and even potentiated the Cd44 expression. From these readouts, we selected the concentration of 5mM for all metabolites, except for SAM, which was added at 0.5mM. We evaluated the C1-MIM of 6 metabolites versus the elimination of SAM (component lethal at high concentrations) and cysteine (component with higher susceptibility to oxidation, but as well is the only one that potentiated the Gfap expression in a dose-fashion). This cocktail represents the C1-MIM_4 with 4 metabolites. For astrocytes, the combination without SAM or cysteine achieved more inhibition of the Gfap marker (FIG. 1J). However, we decided to test with both combinations in the experiments with other cells.

We also observed that the C1-MIM-effect was different in magnitude when applied to mature astrocytes from different origins (from embryonic, postnatal, or adult brain, as observed Gfap inhibitions in FIG. 1I-J). Finally, we evaluated the effect of the addition of a scramble condition of metabolites not related to C1-metabolism, including arginine, creatine, fructose, histidine, leucine, and valine, as scramble cocktail of 6 metabolites, added at 5mM (i.e., with similar concentrations than C1-MIM) (data not shown). Scramble-cocktail did not lower Gfap expression.

Results

Pioneering metabolomic analyses have revealed that different cell types display distinctive metabolic signatures. These studies have primarily focused on steady-state conditions, such as comparisons between stem/progenitor cells and fully differentiated cells^{2,8,9} These experimental designs may miss critical metabolic changes associated with (or potentially driving) the very earliest transitional steps from one cell phenotype to another. Here, the metabolomic changes occurring during the early phases of in vitro cell differentiation were examined in three different multipotent stem cell types (myoblasts (MBs), neural stem cells (NSCs), and mesenchymal stem cells (MSCs)), and uncovered the existence of specific waves of metabolites coupled to the transition of transcriptional programs necessary to drive forward cellular differentiation. Importantly, studies herein demonstrate that metabolites within this wave induce a plasticity window on mature differentiated cells, allowing them to change their identity. Together, these results help to elucidate the metabolome's role during the earliest stages of cell differentiation and unveil the necessity and sufficiency of metabolites to induce cell plasticity and reprogram cell fates.

Immediate metabolomic responses of multipotent stem cells after inducing their differentiation

To identify metabolomic signatures of the earliest stages of cellular differentiation, three well-established, differentiation models: 1) MBs into myofibers, 2) NSCs into astrocytes, and 3) MSCs into chondrocytes were selected and studied. The metabolomes of MBs, NSCs, and MSCs were profiled during their initial steady-state and then at critical time points following induction of cellular differentiation, specifically when the original cells begin to lose transcriptional progenitor cell signatures and acquire markers of early differentiation (FIG. 1A; see discussion SI-1a below). Changes in the expression of selected markers of each progenitor cell and the earliest markers of differentiated derivatives (myofibers, astrocytes, and chondrocytes) were measured. For MBs and NSCs, changes in gene expression were observed as early as 1-3h after differentiation induction, with characteristic markers of differentiation detected after 6-12h. For MSCs, the change of markers was more evident after 24h (FIG. 1B-D). Therefore, a 3-12h time window was considered as an intermediate transcriptional phase for the differentiation of MBs and NSCs; while for MSCs, this window spanned from 6-36h. Moreover, bulk-transcriptomic observations in MBs corroborated an upregulation of metabolic genes in this early time window (data not shown). Therefore, based on gene expression analyses, the time was selected to profile the metabolome engaged in the transitional phase of cells starting differentiation. An ultra-high-performance liquid chromatography-tandem mass spectroscopy (UPLC-MS/MS) platform was then used to comprehensively identify and quantify over 600 metabolites during the intermediate transcriptional phase. Changes in the abundance of each identified metabolite were assessed, and the similarity between samples was computed using both hierarchical cluster analyses and principal component analyses (PCA) (data not shown). While the metabolome of each progenitor separated well from their differentiated counterparts, the data from timepoints during the intermediate phase, at first sight, appeared interspersed without a visible pattern across the different populations (data not shown). Therefore, individual metabolite dynamics were observed. A strategy was developed to associate recurrent patterns by tracing their relative mean abundance over time. For each cell type undergoing differentiation (MBs, NSCs, and MSCs), the recurrent-pattern strategy separates metabolites that do not change over time from metabolites whose abundance displays cumulative, reductive, u-shaped, or wave patterns (FIG. 1E). Wave patterns grouped metabolites whose expression levels increased predominantly during the intermediate transcriptional phase (data not shown). These wave pattern metabolites are of interest, as they represent potential candidates responsible for driving the transitional phase between two distinct cell identities (see discussion SI-lb below). Importantly, enrichment analyses from metabolites exhibiting a waver-like abundance pattern displayed more commonalities across all three-cell type than other abundance patterns (data not shown). This similarity occurs despite differences in the initial metabolomic profiles, transcriptional signatures, culture media, and differentiation time courses between the various cellular systems. Also, supervised K-means clustered analysis was used as an alternative strategy to group the metabolites based on their levels. For each lineage, one cluster displayed similar results to the recurrent-pattern analysis (data not shown). In summary, the patterns of abundance for individual metabolites were profiled and potential shifts in metabolic pathways during the very early stages of cellular differentiation were identified, which concurred with the emergency of transcriptional shifts in cell identity.

A wave of one-carbon metabolism coincides with early transcriptional changes in differentiating cells

Observations from both the recurrent-pattern strategy and K-means clustering revealed that methionine related pathways were present during the early and intermediate transcriptional phases in different cell types. Supporting these observations, more commonalities were found between cell types at the intersections of wave-pattern metabolite enrichments (representing the transition) than at the intersections of reductive or cumulative-pattern enrichments (which represent the steady-states) (data not shown). In all cell lineages explored, methionine and spermine-spermidine metabolism were exclusive to the early-wave-pattern intersection. Methionine participates in a complex interaction of pathways and links to the spermine-spermidine metabolism within the One-carbon (C1) metabolism network²². (FIG. 2A; FIG. 2B). Therefore, we explored shifts in C1-metabolites 3-6h after differentiation induction.

For example, S-adenosylmethionine (SAM), the core product of C1-metabolism^{24,25}, globally increased during the intermediate phase of the cell types tested (FIG. 2C). Overall, across all cell types analyzed, an increase of metabolites associated with C1-metabolism was observed during the transitional phase (FIG. 2B; data not shown). Methionine metabolism is associated with the steady-state of embryonic stem cells (ESCs)²⁶; however, it is not currently associated with the transition-state derived from ESCs. These results suggest that one carbon metabolites may increase during the early transitional phase of a differentiation event, even reaching higher levels than those characterizing in the proper ESC-state. To corroborate this hypothesis, ZHBTc4 ESCs, a well-characterized line were utilized or efficient trophectoderm differentiation²⁷. The results showed that the relative levels of methionine and SAM increased to some extent during the transitional phase throughout trophectoderm differentiation (FIG. 2H-I; discussion SI-1d). Altogether, the results indicate that a conserved Cl-wave coincides with the onset of identity changes for all cell types studied here (FIG. 2D). If this C1-wave is indispensable for cell identity transitions, its disruption may result in steady-state maintenance and prevention or delay in cell identity changes. To determine this necessity, methionine synthase (MTR), the enzyme catalyzing methionine production from homocysteine was knocked down in the three multipotent cell models (MBs, NSCs, and MSCs); then, their differentiation induced. This intervention reduced levels of methionine and evoked increases in select multipotency markers in each cell type, at a time when these markers are highly reduced under normal differentiation conditions (FIG. 2E-G, FIG. 1B-D). Together, these results evidenced that the C1-wave is necessary to proceed with normal cell differentiation. Increases in one-carbon-metabolites in the transitional phase after the initiation of differentiation may be related to their participation in methylation reactions²⁷. Hence, methylation profiles should have an essential restructuration overlapping the transcriptional phase. Thus, reduced representation bisulfite sequencing (RRBS) was performed to explore the dynamics of methylation profiles in NSCs at the initiation of differentiation (data not shown). An increase in methylation was observed as differentiation progressed (FIG. 2J). From a total of 15170 promoters identified, we considered the sites with methylation lower than 25% and higher than 75% to compare the conditions NSC-steady-state and after induction of differentiation (3h and 24h). Next, we detected genes that exclusively have either low or high methylation for each condition and performed functional enrichment analysis. Results showed that during the NSC-steady-state, cell cycle-related loci exhibit less than 25% methylation, but just 3h after inducing differentiation, the main difference occurs at sites related to transcriptional regulation and methylation (FIG. 2L-M). Of note, besides methylation enzymes (e.g., SET and EZH2) and linage specifiers (e.g., Notch and Hes5), key loci associated with one-carbon were differentially methylated at 3h when compared with either the NSC-state or 24h after differentiation (FIG. 2K). Thus, the transient increase in the availability of one-carbon metabolites could serve as a source of metabolic donors for reactions leading on one hand, to the silencing of genes required to maintain original cell identity, and on the other hand, to the activation of cell lineage specifiers via epigenetic regulation. Metabolomic and methylomic levels are less explored in transition-states than the transcriptomic level. Mathematical modeling of the transcriptional dynamics of differentiating NSCs suggests that cells in a transition-state exhibit oscillatory expression peaks in select genes²⁸⁻³⁰.

For example, in NSCs, where Notch/Hes5 signaling controls the differentiation^{31,32}, Hes5 has oscillatory peaks of expression during the transitional phase²⁹. We corroborated Hes5 oscillation, in our NSC-differentiation model (discussion SI-1e). Also, we observed that Matla and Odcl , enzymes participating in the one-carbon network, could possess a similar behavior (FIG. 2L; discussion SI-le). Therefore, not only the levels but the short-term dynamics of the gene expression carry essential information for cell state transitions. In summary, the identified metabolite-wave temporally overlaps with a methylomic and transcriptional dynamism, which may represent a state in which cells may be more susceptible to signals, providing them with the capacity to execute a cell fate decision.

One-carbon metabolites reprogram gene expression and phenotype of differentiated cells

In the above sections, we demonstrated that a conserved C1-wave occurs when a cell exits its steady state and enters the differentiation process. To determine the extent to which artificial C1-metabolite supplementation can facilitate phenotypic transformation (i.e., reprogram cell identity), we treated differentiated steady-state cells with key C1-metabolites, namely methionine, SAM, threonine, glycine, putrescine, and cysteine (data not shown). See discussion SI-1f and SI_4 for details concerning both the culture medium and metabolic combinations. We partially mimicked a C1-metabolite-wave on several types of differentiated cells by culturing them in a medium either without serum or reduced serum and supplemented with C1-metabolite combinations. We refer to these cocktails as C1-Metabolite Induction Medium (C1-MIM). The medium was supplemented with fibroblast growth factor 2 (FGF2) to maintain cell survival only in the case of complete serum elimination (since this will induce cell death after 48h)^{33,34}. Serum reduction (or elimination), as well as the effect of FGF2 alone in the base medium, were evaluated as controls (SI_4). As a quality control, we confirmed that all cell samples lysed for downstream analyses had at least 85% cell viability at the time of collection. The addition of C1-MIM to differentiated mouse cells (including chondrocytes, astrocytes, neurons, and myofibers) or into differentiated human cells (fibroblasts and astrocytes) resulted in morphology changes and consistent decrease in the expression of mature cell markers (FIGS. 2M-P; FIGS. 4A-E). We never detected the expression of the pluripotency gene Oct4, but we observed an increase in the expression of some genes associated with their respective progenitor states (FIG. 4F-J). Collectively, these results demonstrate that treatment with C1-metabolites alters cell identity by reducing markers associated with differentiated cell types and inducing gene characteristics of their respective progenitor states, further supporting the role of one-carbon metabolites in the induction of a transition state.

Supplementation of One-carbon metabolites induces a precursor-like state

We utilized our NSC/astrocyte model to determine the molecular and phenotypic effects of C1-MIM exposure on differentiated cells. In addition to NSCs, astrocytes, and C1-MIM-treated-astrocytes (MIM astrocytes), we included glioblastoma cells (GBs) as a control in this comparison as cancer cells 35 rely on C1-metabolism . Bulk RNA-sequencing (RNAseq) analysis showed that

MIM-astrocytes became more similar to NSCs than to parental astrocytes, and segregated far from GB samples, as observed in the PCA and the Euclidean-distance map (FIG. 3a-b). Moreover, comparisons of Differentially Expressed Genes (DEGs) between NSCs, astrocytes, and MIM-astrocytes revealed that 67.7% of the C1-MIM downregulated genes were highly expressed in astrocytes and downregulated in NSCs. Also, 61% of the genes upregulated by C1-MIM were highly expressed in NSCs (data not shown). We then utilized Gene Set 36,37

Enrichment Analysis (GSEA) , K-means clustering, and Gene Ontology (GO) analysis, for insights into the biological effects of C1-MIM. By GSEA, we obtained the top-20 gene sets upregulated and downregulated by C1-MIM. Most gene sets upregulated (13 of 20 enrichments) associated with the cell cycle, a well-known parameter involved in both cell fate specification and reprogramming³⁸. Gene sets downregulated related to collagen formation processes. The effects on collagen may explain the morphological changes exhibited by these cells (FIG. 3C; data not shown). Orthogonally, the k-means clustering analysis revealed consistent results with GSEA (495 genes associated with cell cycle and 524 genes associated with cell structure and motility were upregulated and downregulated in MIM-astrocytes, respectively) (data not shown; SI_7). Lastly, we analyzed the degree of overlap between DEGs identified in comparisons between C1-MIM-astrocytes, NSCs, and astrocytes. GO analysis of the DEG-overlaps revealed that C1-MIM-treatment: 1) regulates genes involved in metabolic processes, 2) regulates genes involved in the cell cycle, and 3) generates a cell population similar to NSCs but with potentially lesser neurogenic capacity (data not shown; FIG.SA; discussion SI-1g). Bulk-transcriptomic analyses identified essential C1-MIM-effects at a population level, but they can mask differential responses of individual cells. Therefore, we profiled transcriptomes of 9000-14,000 single cells using a 10x Genomics single-cell platform. Gene expression, cell number percentages of astrocyte-markers in each cluster and GO analysis of each cluster in MIM-astrocytes i is in

FIG.SB-D. To provide robustness to our conclusion, we confirmed that the expression levels of astrocyte-and NSC-specific markers agreed between the scRNAseq and bulk-RNAseq datasets, calculating correlation coefficients (FIG. 6A-F; discussion SI-1h). Uniform manifold approximation and projection (UMAP) analysis revealed that the astrocytes group into two clusters, whereas MIM-astrocytes and NSCs group into five and four clusters, respectively, at the same resolution (r=0.2) (data not shown). This separation could imply that MIM-treatment induces a heterogeneity of cell-states like that present in NSCs^{39,40} (data not shown). To identify these potentially shared cell-states between 1) MIM-astrocytes and parental astrocytes or 2) MIM-astrocytes and NSCs, we performed integrated analyses by Seurat (data not shown). This integration identifies anchors across different datasets that represent cells that share biological states based on canonical correlation analysis⁴¹. Integration analysis reveals clearer C1-MIM-treatment effects than our independent UMAP observations. For 1) MIM-astrocytes and parental astrocytes integration, cluster AMO represented most astrocytes (97.3%). This cluster reduced to 6.7% in MIM-astrocytes (i.e., only 6.7% of astrocytes were not affected by C1-MIM). By contrast, cluster AM1 represented 2.86% of astrocytes and 32.51% of MIM-astrocytes. Clusters AM2—AM5 were only identified in MIM-astrocytes, indicating that they acquired more identifiable states under C1-MIM-treatment than untreated astrocytes (data not shown). We then analyzed DEGs between astrocytes and MIM-astrocytes by cluster comparisons for insights into the biological effects of C1-MIM (FIG. 6G-G). In cluster AMO, there were few DEGs, and most downregulated genes were astrocyte markers. Cluster AM1 included a population in which astrocyte markers such as Gfap and Aqp4 expressed at low levels, whereas NSC-precursor genes like Hes5 and Ascll expressed at high levels. Gene ontology analysis of the remaining clusters concluded that genes involved in the cell cycle were relevant to the acquisition of these new C1-MIM-driven phenotypes. By contrast, for 2) MIM-astrocytes and NSCs integration, the populations exhibited a very similar clustering pattern (data not shown; FIG. 7A-F). Despite these similarities, some differences emerged (FIG. 7G-K; discussion SI-li). Of note, cluster NM4 displayed increases in both cell numbers and DEGs compared to other clusters. This cluster was limited in NSCs but strongly enhanced in MIM astrocytes, indicating a feature boosted after C1-MIM-treatment (data not shown). Further, DEGs of cluster NM4 functionally associate to the modulation of the cell cycle (FIG. 7K). Interestingly, MlMastrocytes share this cell cycle feature with the four-factor reprogramed astrocytes⁴², which agrees with the knowledge that the modulation of the cell cycle is one of the immediate responses to the change of cellular states during cell fate specification and reprogramming^{38,43} (data not shown; discussion SI-1j). MIM-astrocytes are more similar to NSCs than to their parental astrocytes; despite this, MIM-cells still display differences with NSCs. Therefore, we characterized their identity based on the cell atlases SciBet and Panglao DB. Results from both training datasets confirm that MIM-astrocytes maintain their neuroectoderm identity (FIG. 8A) and support heterogeneity compatible with radial glial and neuroepithelial cells. Still, they keep a fraction of astrocytic identities (FIG. 3D; FIG. 8B). Therefore, the distinct cell clusters observed in MIM-astrocytes could reflect a heterogeneous population with different commitment levels within this cell lineage (discussion SI-1k). Differences in gene expression between cells may result from a dynamic response to the external C1MIM stimulus. We thus performed a trajectory analysis to address this dynamic response across cells. This analysis revealed the transcriptional re-configuration occurred in MIM-cells over a pseudotime, i.e., rather than dividing MIM-cells into clusters, cells were computational assigned along a continuous path that represents the progression of treatment-dependent change⁴⁴. We found that MIM-astrocytes positioned in five transient states with two potential decision points revealed by a branched trajectory. Branch-1 differentially expressed genes related mainly to ribosome activity and mitochondrial translation processes, while branch-2 differentially expressed genes associated with cell cycle function (data not shown; FIG. 8C-D; discussion SI-11). C1-MIM-effects on the cell cycle may recapitulate the cell trajectory seen with the activation of natural NSCs in adult brains. This trajectory of NSC's activation starts from cells recognized as a subtype of ependymal astrocytes, which differentially upregulate cell-cycle genes in sequential phases to generate populations of neuroblasts³⁹. Therefore, C1MIM-treatment shifted terminally differentiated cells toward an intermediate progenitor-like state, potentially with a similar mechanism to activated adult NSCs.

One-Carbon Metabolites Increase Histone Acetylation and Reconfigure Methylation Patterns

The participation of one carbon-metabolism in methylation is well-documented^{6,24}. We explored methylation-related C1-MIM-effects over our recent transcriptomic data and with other analyses, including the identification of histone modifications, and protein-DNA interactions at promoters of genes of interest. Firstly, at the transcriptomic level, GSEA revealed that one of the top-20 sets of upregulated genes, functionally associated with DNA-methylation in MIM-astrocytes (FIG. 9A). We also examined the expression levels of well-known histone modification genes. We found that MIM astrocytes preferentially exhibited increased expression of methylation-associated genes compared to demethylation-associated genes (FIG. 9B). Next, by mass spectrometry, we measured the changes in the relative abundance of histone modifications induced by the C1-MIM-treatment. We identified 27 histone modifications marks differing between astrocytes and MIM-astrocytes (including acetylation, methylation, and unmarked histones). Most histone acetylation marks were more abundant n MIM-astrocytes than in astrocytes, implying a more relaxed chromatin structure induced by C1-MIM treatment. Whereas histone methylation marks, depending on the site, impact both transcriptional activation and repression (FIG. 9C). We thus considered in detail the site of those methylation marks. Most histone methylation marks in MIM-astrocytes occurred in Histone-3 K27 and K36, which are targets of the methyltransferases Ezh2 and Set, respectively. Besides, according to gene expression readouts, C1-MIM caused higher upregulation of Ezh2 than Set genes (FIG. 9C; also see SI_1m, for a note on Suv39h). Therefore, the repressive mark given by the methylation on H3K27 became the ideal candidate to explore mechanistic insights. Lastly, by chromatin immunoprecipitation of H3K27 in the Gfap-promoter, we concluded that the Gfap-expression loss in MIM-astrocytes is potentially associated with an increase in H3K27-methylation. Conversely, the increase of Hes5 expression may rely on the reduction of that repressive mark in the respective promoter (FIG. 9D-G). Interestingly, prior studies have shown that H3K27-methylation mediated by Ezh2 enzyme has preferential enrichment in pluripotent ESCs at metabolic genes, and it may play an essential role in setting the transcriptional switch leading to metabolic reprogramming⁴⁵. Together, results evidenced that 307 C1-MIM increased the expression of enzymes involved in methylation reactions, restructured the landscape of histones modifications, and favored a relaxed status in the chromatin by increasing acetylation marks.

Suitability of Supplementation of One-carbon Metabolites for Cell Identity Transitions

Finally, we challenged the applicability of C1-MIM with cell fate switch. An attribute of natural NSCs is the ability to form neurospheres. To functionally characterize whether C1-MIM could recapitulate this phenotype, we isolated cerebellar cells from non-neurogenic areas of 1.5-year-old mouse brains and exposed them to C1-MIM. These MIM-astrocytes and untreated controls were then switched into an NSC-medium. Unlike untreated astrocytes, MIM-astrocytes generated neurosphere-like structures within 24h (FIG. 10A). These MIM-astrocyte-derived-neurospheres contained Nestin+and Ki67+cells, indicators of the acquisition of NSC-like traits (data not shown). Moreover, MlMastrocytes-derived-NSCs exposed to a broad differentiation medium, expressed markers for neurons and oligodendrocytes, as well as the recovery of Gfap and other astrocyte markers, indicating gain of multipotency (data not shown, FIG. 10C-C). Although MIM-astrocytes quickly formed neurosphere-like structures, their capacity for sub-culturing was limited to only three passages, which is not the case for natural NSCs. Thus, MIM-astrocytes functionally resembled NSCs, but still, remained distinct from the natural ones (discussion, SI-1n). Despite these limitations, C1-MIM-treatment revealed the induction of a transitional state with enough plasticity for the re-acquisition of some NSCs-traits. Thus, potential applications of C1-MIM may involve paradigms in which boosting a transitional phase may favor cell identity changes, such as the transdifferentiation process. To support this concept, we demonstrated that C1-MIM added before the transduction of MyoD in MSCs (to generate myofibers) or Neurogenin in fibroblasts (to generate neurons) increased and/or accelerated the acquisition of the new identities (data not shown, FIG. 10D-H). Although the effect of C1-MIM-cocktail does not fully recapitulate the natural Cl-wave found during the normal differentiation process. (i.e., the regulation orchestrated by the cell metabolome in the transitional phase) the C1-MIM here described represents a novel direction for inducing cell identity transitions by using metabolites. This work tracks the relationship between specific metabolites and early shifts in cell identity. We uncover a wave of C1-metabolites during the earliest stages of cell differentiation of several multipotent stem cell types. This wave may represent the metabolomic attribute of the transitional state, a phase recognized by several theoretical biology reports^{46,47}, and predominantly explored at the transcriptomic level. C1-metabolites may play a conserved role in a wide range of contexts involving changes in cell identity and cell plasticity. While not being bound by theory, a rapid increase in C1-metabolites may prime the intracellular environment for the deactivation or activation of transcriptional networks, such as pathways that modulate the cell cycle, and provide substrates that enable specific epigenetic modulations⁴⁸⁻⁵⁰, thus favoring a plastic state in which cells are prone to make a fate decision. Indeed, supplying Cl-associated metabolites to fully differentiated cells resulted in a partial conversion to a progenitor-like state.

The observations disclosed herein support that the supplementation of physiological effectors, like the metabolites here described, may represent an alternative strategy to induce cells.
Discussion SI

1a: “Steady-state” refers to the time in which one cell maintains same identity (i.e. with metabolism and transcriptional programs that are the signature of that cell type). Each steady-state requires specific metabolic demands according to its function (i.e., to maintain their homeostasis). For example, the needs of stem cells imply higher anabolic demands compared with differentiated cells and usually are associated with a glycolytic metabolism⁷²⁻⁷⁵.

Among differentiated cells related to oxidative metabolism, a proliferative cell has higher requirements of NADPH and ATP for the biosynthesis compared with a postmitotic one^{8,72}. In this sense, examples of steady-states are the pluripotent stem cells, multipotent stem cells, and every cellular subtype differentiated from each lineage.

Considering cell fate determination as the conjunct activation of transcription factors that will drive a full gene expression program (transcriptional program), when any cell initiates the acquisition of a different identity, invariably occurs a turn-off the original transcriptional program that characterizes it. The signaling that induces such change derives from the niche or medium. Simultaneously to the turn-off of the initial transcriptional program, should occur the turn-on of the new transcriptional program that will identify the subsequent cellular identity. The below sketch represents a cell-type-1 that can receive the signal(s) to start the change in identity. An example of a change in cellular identity occurs during differentiation. However, today we know that the presence of few or even a single transcription factor(s) can drive the conversion of a phenotype into another, giving rise to the paradigms of transdifferentiation and reprogramming. Therefore, the change in cellular identity occurs in different contexts (differentiation, reprogramming, transdifferentiation, natural or artificially induced), but in all of them, the deactivation and activation of transcriptional programs occur. This turn off/on of programs at transcriptional level occurs as a gradual process rather than following a zero-one law. Then, over the progression of this deactivation/activation of programs, there is a time in which both overlap the most, i.e., an intermediate transitional phase. Some studies of theoretical biology have proposed such conceptions, mainly addressing the changes at the transcriptomic level during differentiation between the named steady-state and transition-state46,477⁶. Here, we propose that similar paradigms may occur at other levels, such as the metabolomic one; moreover, the transitional phase could be similar in any other kind of transition between identities, not only limited to differentiation processes.

1b: The metabolome per se is an epigenetic force with potential to regulate gene expression impacting differentiation programs2677However, i) whether the modulation of the metabolism may engage the turn off/on of transcriptional programs or ii) whether a specific metabolome is a requirement for allowing that high transcriptomic dynamism, requires more scrutiny. Therefore, we explored the metabolome associated to an intermediate phase because in this phase occurs a major dynamism derived from the deactivation/activation of transcriptional programs that characterize the identity of the cell types located at each extreme of the process (steady-states). Along all processes there is a transcriptional program going on. Once steady-state is reached (committed), the transcriptional program is supportive for the homeostasis, specific for each cell type, and different from the dynamic of the intermediate transcription. Similarly, in different cells harboring steady-states (pluripotent, multipotent, or fully differentiated) there is a metabolome that characterizes them according the homeostatic demands proper to the physiology of each cell type. Contrary, when cells are induced to change their identity, they may require specific metabolic demands to support both the essential homeostasis and the process associated with changing the identity. For example, we can visualize the shift from oxidative-phosphorylation to glycolysis occurring during reprogramming^{74,75,78}, this shift compares the metabolisms of steady-states; and to allow this shift to occur, potentially a specific metabolome may be required.

Recognizing the difference between a metabolome supportive of a steady-state versus a metabolome supportive of a change's process, allow us to distinguish the importance of the pattern of abundance of the metabolites In the model sketched (FIG. 1F), the importance of the pattern together with the abundance of the metabolites is represented. The hypothetical metabolite-2 is higher in their levels than metabolite-1. However, the levels of metabolite-2 can be a consequence of the loss of the original identity (i.e., metabolite-2 could be part of the signature of the initial steady-state, and because this state starts to be repressed, the levels of metabolite-2 decay). Conversely, metabolite-1 and -3 show a spike or bell-like increase during an early time after trigger of the change of identity (this trigger is an independent external signal), which means that those metabolites which increase their levels particularly in that window, may play a role in the higher transcriptional activity representing the turn on/off of cellular identities.

On the other hand, it is probable that metabolites that are exclusively absent in that period have an implication as well. However, this study focused only on those that increase their levels because i) from the recurrent-pattern strategy, the bell-wave or spike occurred in a robust way (timing) in all the cell types tested, while the u-shaped represents a more gradual and less consistent change compared with the opposite spike; and ii) experimentally, an increase of metabolites can be easily manipulated. Other patterns, like reductive and cumulative, represent the exclusive signatures of the initial and final phenotypes, respectively. In this respect, u-shaped metabolites represent part of the signature that is shared by both phenotypes. Different types of cumulative, only reflect different rates of intracellular accumulation, as the acquisition of the final phenotype is occurring. Finally, we also found bell patterns or waves located after being increased the markers of the second phenotype, potentially associated with the maturation of cell-specific characteristics. These late-bells do not overlap with the transitional phase and consequently were not conserved in time and composition between different cell types.

1c: K-means clustering is a robust analysis, but it reduces or amplifies the importance of changes based on the behavior of the whole; thus, it underestimates some trends79$° . In this context, we settled the Recurrent Pattern

Classification strategy (RPC-strategy), which is advantageous for the easy grouping and visualization of individual dynamics of metabolites. RPC-strategy aims to identify even subtle increases in metabolite's levels in specific time windows, which may not appear as evident otherwise, and that could have the potential to cause drastic effects. These caveats should be kept in mind when interpreting the data from clustering analysis vs. RPC-strategy, as they weigh the aspects of the data differently. The impact of discrete increases in molecules can be exemplified with the cell culture in vitro, where there is a range of compounds and small molecules added (some at range picomolar, nanomolar, others at range millimolar, etc.). It is common to observe that some small molecules added even in a transient period at picomolar concentrations may drive more potent effects that other compounds present at higher concentrations even during sustained periods. Thus, RPC-strategy allowed us to consider metabolites in the transitional phase, even with small increases.

1d: Methionine and SAM levels were indirectly measured by fluorometric and colorimetric assays, respectively. These assays gave a good idea of proportions between conditions but may return inaccurate concentrations depending on the standard curve. Further comprehensive metabolomic studies of the dynamics of the transitional phase of ESCs will be ideal for the future. With the current described approaches, we observed at 12h only a slight increase of methionine; this metabolite is the precursor of SAM, which showed higher levels at 18h (compared with 12h); therefore, the reduction of methionine at 18h could be a consequence of the increase in SAM at that time, while methionine potentially could show higher levels at an earlier time point than the 12h measured in this study.

1e: Theoretical studies propose that the gene expression in cells close to a bifurcation boundary for fate decision have a transient period of oscillatory peaks of expression of specific genes. This phenomenon has been studied in the transitional phase of NSCs. One of the first responders of NSC-differentiation is Hes5. Discoveries published by Manning et al. 29 shown that neural progenitors have Hes5 fluctuations, where Hes5 periodically spikes as cells transit to differentiation. The paper, focused on transcriptional changes, suggested that the control of cell state is an oscillatory behavior occurring during a few hours. Therefore, not only changes in the gene expression levels are essential, but the short-term dynamics of the gene expression carry essential information for cell state transitions. Our model may represent parallelism in this dynamism but at the metabolomic level. We observed a potential temporal/behavioral connection between the occurrence of a fast oscillation (wave) of C1-metabolites and Notch signaling. In agreement with former observations, we found an oscillatory peak of Hes5 expression immediately after inducing the differentiation process (FIG. 2L). Then, we explored whether any of the C1-metabolism-enzymes could show similar behavior. Among those, we observed a discrete oscillation of Odcl , enzyme limiting of the polyamine metabolism, and large oscillatory spikes on Matl a expression, one of the main enzymes involved in the methionine cycle. Here the oscillatory peak in gene expression was determined by rt-PCR, thus, at the level of different pools of cells crossing by the same time, and it was observed in those referred genes, but not in others (such as Mtr or Ahcy evaluated simultaneously). Intriguingly, the Matla peak timely fits with the oscillatory peak of Hes5, and they seemed potentially codependent. Briefly, we did the knockdown Matla with siRNA [250nM] in NSCs; after 24h, we induced them to differentiate. As expected, we found the early peak of Matl inhibited (in 45.1% ±16.7, 121 n=10), and unexpectedly we found that the Hes5 peak was also inhibited in 18.8% ±3.02 (n=6); therefore, it is tempting to speculate a potential codependence of those elements in a context of normal differentiation. While there is no report of the signaling connecting those both elements, by doing a STRING prediction analysis, we observed that DNA-methyltransferases are the putative link between those components (Hess and Matl), which makes sense with the known role of C1-metabolites like SAM and Methionine as players in epigenetic changes. Future studies addressing the challenge of generating a Multi-Omics integrationsi (i.e., genomics +methylomics +transcriptomics +proteomics +metabolomics) could clarify that connection and the operational mechanism of the transitional phase.

1f: For our purposes, the supplementation of some metabolites to feed the C1-network is enough rather than the full replication of the early wave/bell-shaped metabolome. The methionine and polyamine metabolisms are those representing the commonality between the top enriched pathways for each intermediate transcriptional phase. Still, as expected, there are other pathways specific per cell lineage. Therefore, the full replication of the wave, although it might be the ideal case (in a lineage-specific setup), is not practical for different cell types. However, C1-metabolites, although present with different enrichment values per cell type, they appear constant between different cell types going through the transitional phase. We therefore selected target metabolites that may synthetically feed the Cl-wave, starting with methionine and S-adenosylmethionine (SAM), because they are considered central C1-metabolites, which in turn interact with the polyamine biosynthesis pathway, where putrescine is the primary precursor (and over which SAM may act). Another reason to select putrescine is the finding of increased expression of Matl (an enzyme produced after the step of catalyzation of putrescine to spermidine). Besides, the cysteine-glycine-threonine cycle interconnects with the methionine cycle and feeds the transsulfuration pathway leading the glutathione metabolism“”. The limiting step for the glutathione cycle is the pool of cysteine FIG. 2B). On the other hand, threonine regulates SAM concentrations, and it is known its influence on the differentiation of ESCs⁸². Overall, we selected the C1-metabolites based on the potential relevance of its participation in the C1-network.

For initial tests, we used astrocytes because, in the NSC-lineage, the C1-metabolism had higher lsignificance, according its P-value, compared with MBs and MSCs. Details about the customization of the cocktail are in SI_4. We tested our selected components, one by one and in a range of doses from [0.025 to 10mM]. The effect of single metabolites (if any) always was lesser than that found when we used a combinatory setup (either four metabolites, MIM(4): methionine, threonine, glycine, putrescine; or six metabolites, MIM(6): the four previous, plus SAM and cysteine). From the individual test of metabolites, we observed that cysteine potentiates Gfap expression rather than inhibit it, while S-adenosylmethionine is toxic in the millimolar range (SI_4); therefore, the supplement was tested in the presence or absence of those metabolites as well. In agreement with these results, the elimination of cysteine from the cocktail repressed, even more, the Gfap expression, while the elimination of SAM did not affect such reduction. Finally, the combined withdrawal of cysteine and SAM had the best repression of Gfap (SI_4). In the case of differentiated neurons, those cells only 163 tolerated the MIM(4) treatment, showing a similar reduction of their mature differentiation markers β-164 Tubb3 and Map2 (FIG. 20). Additionally, we tested a scramble control composed of 6 metabolites that do not relate directly with the one-carbon cycle (SI_4). Gfap expression was increased rather than inhibited in the scramble condition.

Because the increase of C1-metabolites occurs in a context where the initial cellular identity (MBs, NSCs or MSCs) is induced to differentiate by external signals (distinct media per lineage, a conserved Cl-wave might be supportive for the transition rather than the trigger of that. Therefore, in a context promoting a steady-state (such as that maintained by high concentrations of serum), it is expected do not observe any significant change in identity as we corroborated. This may explain the fact that in the conditions in which we added FGF2 to support the cell survival in serum-free media, it acted as a master inductor to redirect the phenotype in a metabolomic environment propitious for the change and created by C1-MIM. This agrees with the experiments shown in FIG. 10D-H, where the master-inductors are the molecules MyoD or NGN1/2 that lead the transdifferentiation process, while C1-MIM may contribute to the intracellular dynamics necessary that facilitate taking a bifurcation or cell fate decision.

The reduction of serum and especially the combination with an additional factor is the best schema to see C1-MIM effects. Of note, methods to do chemical reprogramming of MEFs are usually developed in a serum-free defined media⁷⁸′⁸³.

To better identify whether after the addition of C1-MIM is possible to acquire a new phenotype, we performed sustained feeding with the cocktail every other day for five days. The reason for this schema is the observation that even in normal differentiation (when the Cl-wave occurs, shortly after starting the induction), the early time points do not have a new discernible cellular identity yet. The acquisition of a new phenotype usually can be fully characterized after several days (even weeks, depending on the cell type) after inducing the differentiation. In this context, we supplied Cl MIM several days to identify whether a new cell type emerged. However, consider that the changes induced by the supplementation of this cocktail occur shortly after its addition, as evidenced by the tracking of gene expression of critical markers in MIM-astrocytes (FIG. 9D,F). As final remark regarding C1-MIM composition, we are aware that the concentrations of metabolites here supplemented are still far to be optimal to replicate in the best way the Cl-metabolome. Here, we used equimolar concentrations (on 5 of 6 metabolites) that may saturate the system, and potentially a combination of concentrations for each metabolite could be more effective.

Of note, we measured the intracellular levels of the metabolites in cells after being exposed to C1-MIM, and we found that the levels of metabolites increase in 1.1 to 11 times, keeping a range still considered physiological. Although the concentrations of C1-metabolites still may require further customization, the prevalent finding of our study is the occurrence of Cl-wave at the onset of a change of transcriptional identity. Future research could address the standardization of the ideal concentrations of metabolites to boost the Cl-metabolome adapted to each cell type.

lg, In our study, we comparatively evaluated data derived from bulk and single-cell RNA sequencing. From both sets, we revealed similar tendencies in downregulated/upregulated genes (of note, generated from independent samples for each respective assay). From the analyzed overlaps of the DEGs between MIM-astrocytes, NSCs, and astrocytes comparisons, we detected 1215 genes exclusively higher expressed in MIM-astrocytes compared to NSCs (FIG. 5A).

The enrichment analysis of this group shows metabolic-related processes, which may be a consequence of the boosting of the cycling of metabolites across their pathways following the artificial supplementation of them. In the same line, here is a group of genes that are higher expressed in NSCs compared to MIM-astrocytes (n=1001); the enrichment analysis of this group reveals more neurogenic capacity in the natural NSCs compared to MIM-astrocytes (FIG. 5A). Also, there are groups of genes highly expressed in astrocytes and MIM-astrocytes, but not in NSCs, and inversely (FIG. 5A). From these comparisons, we inferred that 973 shared genes are upregulated in NSCs vs. astrocytes, but also upregulated in MIM-astrocytes vs. NSCs. This group enriched for cell cycle processes FIG. 5A).

1h, Most of the correlation analyses using log fold change of gene expression between scRNAseq and bulk-RNAseq from astrocytes vs. MIM-astrocytes comparison, correlated very well except Cluster-AM3 (FIG. 6C, Pearson r=0.110, p-value=0.069).

li, When the populations of NSCs and MIM-astrocytes integrate, they largely overlap, but they are not identical (data not shown; FIG. 7A-K). From cell number and percentage comparisons between clusters, we found that in MIM-astrocytes Cluster-NMO and -NMI, the cell number and percentage is lower, while Cluster-NM2, -NM3 and -NM4 the cell number and percentage is higher compared to NSCs 223 (FIG. 7A-K). Then, from DEGs analysis from the clusters of this integration, we found that all the clusters in MIM present downregulated genes expression pattern but not upregulated comparing with NSCs, except by NM4 cluster. Cluster-NM4 not only has a relatively more downregulated number of genes but also has the highest upregulated number of genes compared with other clusters. The correlation analysis showed again that gene expression changes of NM4 (fold changes) are similar in single-cell and bulk-RNAseq. Further, GO analysis of those DEGs showed that downregulated genes of MIM are most associated with central nervous development (like cluster NMO, NM3, and NM4) while upregulated genes of MIM are most related with amyloid fibril formation (like NMO, NM1) (FIG. 7A-K).

1j, Nakajima-Koyama et al. 42 published a study on the reprogramming of mouse astrocytes with the four Yamanaka factors. They detailed a transcriptomic characterization on those reprogrammed astrocytes, showing that astrocytes are reprogrammed through an NSC-like state. Because MIM-astrocytes showed NSC-like characteristics, we compared the DEG upregulated in the intermediate state found by Nakajima-Koyama et al., vs. the NSC-like-state of MIM-astrocytes. We found that those populations shared 446 genes upregulated (data not shown). KEGG-enrichment of those genes points to cell cycle-related processes. Of note, from gene set enrichment analysis (GSEA) on MIM-astrocytes-bulk-transcriptomics, considering the top 20 gene sets upregulated by C1-MIM, the majority (13 of 20 enrichments) were associated with the cell cycle (SI_8). While, from the scRNAseq integration analysis we found that cells from cluster AM2 notably expressed cell cycle-related genes such as Prcl, Cdkl , Cenpe, Cdca8, Cdc20, Chekl, Cenpf, Mki67, and others (FIG. 6H,N); while gene ontology analysis of cluster NM4 DEGs functionally enriched for genes involved in modulating the cell cycle (FIG. 7K).

1k, Studies about single-cell transcriptome of natural NSCs394° demonstrated that NSCs appears as a heterogeneous spectrum of progenitors in different stages of commitment between activated and committed to differentiation. Similarly, the clustering observed in MIM-astrocytes, reveals distinct cell states, like those occurring in natural NSCs, potentially representing different levels of commitment.

1l, C1-MIM-treatment potentially induces distinct cell states that could be represented by trajectory analysis447^{44,70}. One rationale for this kind of analysis is the consideration of a potential asynchrony found in a population of cells captured at the same time after MIM-treatment, which may create difficulty in observing cells that are in the transition from one state to the next state. Results showing that the MIM population has five cell states and two branches potentially imply that from the pool of cells, those do not respond to the treatment in identical fashion (data not shown; FIG. 8C-D).

1m, Despite the high expression of Suv39h-1-2 (FIG.9B), the associated modification H3K9me3 was less evident for MIM-cells (FIG. 9C) compared to modifications on Histone-3 K27 and K36 (which in turn, are targets of the methyltransferases Ezh2 and Set, respectively).

1n, Observations about the transient plasticity in MIM-astrocytes include their capacity to form neurosphere-like structures when exposed to a medium for NSCs proliferation. Natural NSCs are characterized by functionality due to the absence of exclusive markers. Functional identification of bona fide NSCs includes three properties: proliferation, self-renewal, and multipotency, deeply reviewed by Gil-Perotin et al.⁸⁴ MIM-treated phenotype acquired is a good fit in this characterization. Besides to the proliferation and multipotency proofs shown in FIG. 10A-H, we performed a low-density assay as a way to track self-renewal. For this kind of assays, most of the studies have seeded densities ranging from 5 to 50 cells/μL⁸⁵⁻⁸⁷. We seeded MIM-astrocytes-derived-NSCs at a density of1cell/μL in 96-well 270 plates with 100 μL of NSC medium per well; after one month, we found that 2.7% ±0.6 of the wells presented a cell population growing (n=3 plates). This percentage is similar to the reported for natural NSCs⁸⁵. This quantification was performed during the first passage of MIM-astrocytes-derived-NSCs because we observed as a relevant difference to natural NSCs that cells do not proliferate well after the third passage. Another difference is related to multipotency. β-Tubb3 was detected early after shifting the MIM-astrocytes into NSC-medium, which suggests a rapid acquisition of neuronal progenitors. However, after proceeding with the differentiation, despite the acquisition of the maturation markers such as Neuron-Specific Enolase, Synaptophysin, and Map2, we did not observe the survival of the neurons after one week, this may be a problem related with the intrinsic capacity of the MIM-neuron derived cells (as suggested by transcriptomic profile) but as well the necessity of an in-depth optimization of an appropriate protocol for neuronal differentiation starting from those cells (which may be a goal outside of the scope for this paper). These outcomes are in agreement with our transcriptomic data, which suggested a reduced neurogenic capacity of MIM-astrocytes when compares with NSCs (FIG. 5A).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

1. A cell culture medium composition for modulating cellular steady state comprising at least two C1-metabolites.

2. The kit or cell culture media composition of claim 1 the composition comprising C1-metabolites selected from the group consisting of methionine, SAM (S-adenosyl methionine), threonine, glycine, putrescine, and cysteine, in amounts effective to induce re-programming of differentiated or partially differentiated eukaryotic cells into progenitor like cells.

3. The composition of claim 1, comprising at least three C1metabolites (C1 MIM).

4. The composition of claim 1 wherein the CI-MIM comprise methionine, threonine, glycine, and putrescine.

5. The composition of claim 4 further comprising SAM or cysteine, wherein the composition comprising SAM optionally comprises SAM at less than 0.5 mM.

6. (canceled)

7. The composition of claim 1, comprising cell culture medium, optionally, wherein the cell culture medium is serum free or comprises less than 3% serum.

8. (canceled)

9. The composition of claim 7, comprising FGF.

10. The composition of claim 1, further comprising phosphate buffered saline (PBS), Dulbecco's Modified Eagle's Medium (DMEM), Minimal Essential Medium (MEM), Basal Medium Eagle (BME), Roswell Park Memorial Institute Medium (RPMI) 1640, MCDB 131, Click's medium, McCoy's 5 A Medium, Medium 199, William's Medium E, insect media such as Grace's medium, Ham's Nutrient mixture F-10 (Ham's F-10), Ham's F-12, a-Minimal Essential Medium (aMEM), Glasgow's Minimal Essential Medium (G-MEM), Iscove's Modified Dulbecco's Medium, Neurobasal media, DMEMF12 or MEM Alpha.

11. A kit comprising the composition of claim 1.

12. A method of dedifferentiation of partially or completely differentiated cells to obtain C1 metabolite-induced de-differentiated cells (MIM cells) with progenitor-like characteristics, the method comprising:
culturing the cells to be induced with the composition of claim 7 for a period of time effective to induce de-differentiation, wherein de-differentiation is determined as a reduction in the expression of at least one mature cell marker expressed in the when compared to partially or completely differentiated cells, when compared to cells cultured in the composition of claim 7.

13. The method of claim 12, wherein the differentiated cells are selected from the group consisting of multipotent stem cells, cells of hematological origin, cells of embryonic origin, skin derived cells, fibroblasts, adipose cells, epithelial cells, endothelial cells, mesenchymal cells, parenchymal cells, neurological cells, and connective tissue cells.

14. The method of claim 13, wherein the cells to be induced are selected from the group consisting of fibroblasts, chondrocytes, adipose-derived cells, neural derived cells and intestinal epithelial cells.

15. The method of claim 12, wherein the cells are not transfected to express any of Oct4, KLF4, SOX2, C-Myc or NANOG.

16. The method of claim 13, further comprising isolating the MIM cells.

17. The method of claim 16, wherein the MIM cells are isolated using a progenitor marker.

18. C1 metabolite-induced de-differentiated cells (MIM cells) obtained by the method of claim 12, wherein the cells show a reduction in the expression of at least one mature cell marker expressed in the differentiated or partially differentiated cells when compared to untreated partially or completely differentiated cells.

19. (canceled)

20. The cells of claim 18, obtained from: (a) astrocytes, wherein the MIM -Cells show decreased expression of Glial fibrillary acidic protein encoding gene, Gfap and Cd44 (Cluster of differentiation 44), when compared to astrocytes; (b) chondrocytes wherein the MIM-Cells show decreased expression of the chondrocyte markers Aggrecan, and collagen type II (Col2) when compared to chondrocytes; (c) neurons, wherein the MIM-cells show decreased expression of Map2 and beta-III-tubulin; and/or (d) obtained from fibroblast, wherein the MIM-cells show decreased expression of collagen type I alpha 2 chain encoding gene, Col1A2.

21. (canceled)

22. (canceled)

23. (canceled)

24. The cells of claim 18, a) wherein the cells show upregulated expression of genes associated with progenitor states, wherein the gene is selected from the group consisting of cMyc, Ascll, SOX2, SOXS, Nestin, CD133, MyoD, and Pax7; or (b) wherein the cells show an increase in the expression of at least one cell cycle related gene and/or reduction in the expression of at least gene related to collagen formation.

25. The cells of claim 24, wherein the cells do not express Oct4, Klf4, and/or Nanog.

26. (canceled)

27. The cells of claim 18, obtained from: (a) astrocytes, wherein the MIM-Cells show increased expression of at least one methylation-associated gene selected from the group comprising Setd1a, Km2a, Km2b, Km2d, Smyd2, Suv39h1, Suv39h2, Ehmt2, Ehmtl, Sstdbl, Ezh2, Setd2, Nsdl, Nsd2, and Ash1L;
optionally wherein the cells show increased histone acetylation, optionally wherein the histone acetylation is as at one or more sited selected from H2A K5, H2A K9, H3 K14, H3 K18, H3 K23, and H 3.1 K27; or (b) astrocytes, wherein the MIM-Cells can form neurosphere-like structures, Nestin+and/or Ki67+neurospheres, or combinations thereof when cultured in NSC medium;
optionally wherein the MIM-Cells can be differentiated into neurons, oligodendrocytes, and/or astrocytes.

28. (canceled)
